(19) United States
(12) Patent Application Publication
(10) Pub. No.: US 2022/0265776 A1
(43) Pub. Date: Aug. 25, 2022

(54) COMBINATION

(71) Applicant: GENESIS PHARMA SA, ATHENS (GR)

(72) Inventor: George VOGIATZIS, Athens (GR)

(21) Appl. No.: 17/624,690

(22) PCT Filed: Jul. 9, 2020
(86) PCT No.: PCT/EP2020/069343

§ 371 Date: Jan. 4, 2022

(30) Foreign Application Priority Data
Jul. 9, 2019 (GR) 20190100288

(51) Int. Cl. A61K 38/26; A61K 31/64; A61K 31/585; A61K 31/575; A61K 31/4353; A61K 45/06; A61P 25/28; A61P 9/10

(52) U.S. Cl. CPC A61K 38/26; A61K 31/64; A61K 31/585; A61P 9/10; A61K 31/4353; A61K 45/06; A61P 25/28; A61K 31/575

(57) ABSTRACT

The present invention provides a combination comprising (a) a sulfonylurea; and (b) at least one of the following components: (i) an insulin modulator, and (ii) an aldosterone antagonist, for instance a combination comprising (a) glibenclamide, or a structural or functional analogue thereof; and (b) at least one of the following components: (i) exenatide, or a structural or functional analogue thereof, or a pharmaceutically acceptable salt thereof; and (ii) potassium canrenoate, or a structural or functional analogue thereof. Said combinations are suitable for the treatment of stroke and other neurodegenerative disorders and for treating and/or preventing ischemia and/or reperfusion injury in various vital organs, including the brain and the heart. Further aspects of the invention relate to pharmaceutical products and pharmaceutical compositions comprising said combinations according to the invention, and methods of treatment using the same.

### FIELD OF INVENTION

The present invention provides a combination suitable for the treatment of stroke and neurodegenerative disorders, and for treating and/or preventing ischemia and/or reperfusion injury in various vital organs, including the brain and the heart.

Further aspects of the invention relate to pharmaceutical products and pharmaceutical compositions comprising said combinations, and methods of treatment using the same.

### BACKGROUND

Stroke is caused by lack of blood flow in the brain (ischemic stroke) or by bleeding in the brain (haemorrhagic stroke) and both conditions result in brain cells death. It is the second most important cause of death globally, accounting for about 6 million deaths in 2016 according to the World Health Organisation. There were 13.7 million new stroke cases and 80.1 million prevalent cases of stroke globally in 2016, according to the Global Burden of Disease study (Johnson C O, et al. Lancet Neurol. 2019; 18: 439-58). The high burden of stroke worldwide suggests that primary prevention strategies are either not widely implemented or not sufficiently effective. Guidelines are available for the management of acute ischemic stroke (Powers W J, et al. Stroke. 2018; 49: e46-e99). Interestingly, the guidelines conclude that at present, no pharmacological or non-pharmacological treatments with putative neuroprotective actions have demonstrated efficacy in improving outcomes after ischemic stroke, and therefore, other neuroprotective agents are not recommended. Guidelines also exist for the management of haemorrhagic stroke; however, they do not recommend therapies for the management of the neurodegenerative consequences of haemorrhagic stroke other than rehabilitation (Hemphill J C 3rd, et al. Stroke. 2015; 46: 2032-2060).

The data above clearly indicate that presently effective pharmacologic treatments of ischemic or haemorrhagic stroke are lacking and that there is a need for treatments that are neuroprotective in patients with stroke. An effective treatment of the reperfusion injury that is associated with stroke has the potential to offer neuroprotection. However, attempts so far to develop effective neuroprotective treatments for stroke patients, which are based on decreasing the reperfusion injury have not been successful (Savitz S I, et al. Stroke. 2017; 48: 3413-3419; Patel R A G, et al. Prog Cardiovasc Dis. 2017; 59: 542-548). Previous studies in the area of cardioprotection and reperfusion injury have revealed the surprising finding that combinations therapies, not indicated for the treatment of cardiovascular disorders, if used at dose levels that are lower than the ones indicated for these other conditions can result in significant synergistic effects that protect against myocardial reperfusion injury (WO 2017/077378; U.S. Pat. No. 10,172,914; Genesis Pharma SA).

Treatments showing neuroprotective effects are expected to be useful in the treatment of neurodegenerative disorders. Neurodegenerative disorders are due to a progressive loss of structure or function of neurons, which eventually leads to the death of neurons. They include diseases such as Parkinson's disease, Alzheimer's disease, Huntington's disease, amyotrophic lateral sclerosis and vascular dementia that are currently incurable. Alzheimer's disease showed the highest increase (46.2%) among the neurological disorders globally over the period 2007-2017, reaching 2.51 million deaths in 2017 (Roth G A, et al. Lancet 2018; 392: 1736-88). In the same study, Parkinson's disease also showed an increase of 38.3% accounting for 340,600 deaths globally in 2017. Amyotrophic lateral sclerosis has a prevalence of 5.40 in Europe, 3.40 in the USA, and 2.34 in Asia per 100,000 population (Marin B, et al. Int J Epidemiol 2017; 46: 57-74). Huntington's disease is a hereditary neurological disorder that is considered a rare disease with a prevalence ranging from 0.4 per 100,000 population in Asia to 7.3 per 100,000 population in North America (Rawlins M D. Neuroepidemiology. 2016; 46: 144-53). Vascular dementia is dementia caused by problems in the supply of blood to the brain, typically a series of minor strokes, leading to worsening cognitive decline that occurs step by step. The term refers to a syndrome consisting of a complex interaction of cerebrovascular disease and risk factors that lead to changes in the brain structures due to strokes and lesions, and resulting changes in cognition. Vascular dementia is the second most common form of dementia after Alzheimer's disease (AD) in older adults (Battistin L, (December 2010), Neurochemical Research 35 (12): 1933-8; “Vascular Dementia: A Resource List”). The prevalence of the illness is 1.5% in Western countries and approximately 2.2% in Japan. It accounts for 50% of all dementias in Japan, 20% to 40% in Europe and 15% in Latin America. 25% of stroke patients develop new-onset dementia within one year of their stroke. One study found that in the United States, the prevalence of vascular dementia in all people over the age of 71 is 2.43%, and another found that the prevalence of the dementias doubles with every 5.1 years of age (Plassman B L, (2007), Neuroepidemiology. 29 (1-2); Jorm A F (November 1987), Acta Psychiatrica Scandinavica. 76 (5): 465-79). Currently, there are no medications that have been approved specifically for the prevention or treatment of vascular dementia. The currently approved therapies for Alzheimer's disease provide only modest benefits (Atri A. Med Clin North Am. 2019; 103: 263-293). A number of pharmacologic treatments are available for managing the motor and non-motor symptoms in Parkinson's disease, but they are essentially symptomatic treatments and eventually induce dyskinesias while none of them provides neuroprotection (Chaudhuri K R, et al. Parkinsonism Relat Disord. 2016; 33 (Suppl 1): S2-S8). There are currently only two approved two drugs (riluzole and edaravone) that slow down the progress of amyotrophic lateral sclerosis, albeit modestly, and there is no approved therapy for Huntington's disease.

There is therefore a clear need for further and better treatments that offer neuroprotection, particularly in the context of treating stroke, cerebral reperfusion injury and certain neurodegenerative diseases.

### STATEMENT OF INVENTION

The present invention provides combinations which are neuroprotective and suitable for the prevention or treatment of cerebral reperfusion injury, stroke and other disorders/diseases where neuroprotection is desirable. Advantageously, the presently claimed combinations and other aspects of the invention provide a treatment which is more efficacious and provides superior clinical outcomes compared to therapies that employ a single active pharmaceutical agent.

A first aspect relates to a combination comprising:

(a) a sulfonylurea; and
(b) at least one of the following components: (i) an insulin modulator, and (ii) an aldosterone antagonist.

A second aspect relates to a combination comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

A third aspect relates to a pharmaceutical composition comprising:

(a) a sulfonylurea; and
(b) at least one of the following components: (i) an insulin modulator, and (ii) an aldosterone antagonist; and
a pharmaceutically acceptable carrier, diluent or excipient.

A fourth aspect relates to a pharmaceutical composition comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof; and
a pharmaceutically acceptable carrier, diluent or excipient.

A fifth aspect relates to a pharmaceutical product comprising:

(a) a sulfonylurea; and
(b) at least one of the following components: (i) an insulin modulator, and (ii) an aldosterone antagonist.

A sixth aspect relates to a pharmaceutical product comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

A seventh aspect relates to a combination or a pharmaceutical composition or product as defined above for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection, for example, against neurotoxic drugs.

An eighth aspect relates to a pharmaceutical product as defined above for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection, for example, against neurotoxic drugs, wherein the components are for administration simultaneously, sequentially or separately.

A ninth aspect relates to a method of treating and/or preventing one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection, for example, against neurotoxic drugs, said method comprising simultaneously, sequentially or separately administering to a subject in need thereof:

(a) a sulfonylurea; and
(b) at least one of the following components: (i) an insulin modulator, and (ii) an aldosterone antagonist.

A tenth aspect relates to a method of treating and/or preventing one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection, for example, against neurotoxic drugs, said method comprising simultaneously, sequentially or separately administering to a subject in need thereof:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

An eleventh aspect relates to the use of:

(a) a sulfonylurea; and
(b) at least one of the following components: (i) an insulin modulator, and (ii) an aldosterone antagonist;
in the manufacture of a medicament for the treatment and/or prevention of one or more of ischemia and/or reperfusion injury, stroke, a neurodegenerative disease, neonatal asphyxia, cardiac arrest, cardiogenic shock and acute myocardial infarction, or for providing cardioprotection against cardiotoxic drugs, or for providing neuroprotection, for example, against neurotoxic drugs.

A twelfth aspect relates to the use of:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
in the manufacture of a medicament for the treatment and/or prevention of one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection, for example, against neurotoxic drugs.

A thirteenth aspect relates to the use of a combination comprising:

(a) a sulfonylurea; and
(b) at least one of the following components: (i) an insulin modulator, and (ii) an aldosterone antagonist;
for treating and/or preventing ischemia and/or reperfusion injury in an ex vivo organ prior to or during transplantation.

A fourteenth aspect relates to the use of a combination comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for treating and/or preventing ischemia and/or reperfusion injury in an ex vivo organ prior to or during transplantation.

### DETAILED DESCRIPTION

The preferred embodiments set out below are applicable to any of the above-mentioned aspects of the invention as appropriate.

As used herein, a structural analogue, also known as a chemical analogue, is a compound having a structure similar to that of another compound, but differing from it in respect to a certain component. It can differ in one or more atoms, functional groups, or substructures, which are replaced with other atoms, groups, or substructures. A structural analogue can be imagined to be formed, at least theoretically, from the other compound. Structural analogues are often isoelectronic.

As used herein, functional analogues are chemical compounds that have similar physical, chemical, biochemical, or pharmacological properties to that of another compound. Functional analogues are not necessarily structural analogues with a similar chemical structure.

### Sulfonylureas

The combinations of the invention comprise a sulfonylurea as an essential component.

Sulfonylureas are a class of oral hypoglycaemic agents that are mainly used in the management of type 2 diabetes and certain forms of monogenic diabetes. They reduce blood glucose levels by stimulating insulin secretion from pancreatic β-cells. Their primary target is the sulfonylurea receptor (SUR1) subunit of the ATP-sensitive potassium (KATP) channel in the β-cell plasma membrane (Proks P, et al. Diabetes. 2002; 51 (Suppl 3): S368-76; Gribble F M and Reimann F. Diabetologia. 2003; 46: 875-891).

Sulfonylureas are traditionally classified into two generations, consistent with the time of their introduction in the clinic, with differences mainly in their disposition, which allows for a less frequent administration of the drugs that belong to the second generation (Sola D, et al. Arch Med Sci. 2015; 11: 840-8):
the first-generation includes chlorpropamide, tolbutamide, acetohexamide, carbutamide, glycyclamide, tolhexamide, metahexamide, and tolazamide; however, these are no longer used in clinical practice;
the second-generation includes glibenclamide (glyburide), glibornuride, gliclazide, glipizide, glimepiride, gliquidone, glisoxepide and glyclopyramide. Modified/extended release formulations exist for some of the second-generation sulfonylureas (gliclazide, glipizide).

Current guidelines recommend the use of second generation sulfonylureas as second-line therapy in combination with metformin, when inadequate control was achieved with metformin alone, and second generation sulfonylureas may also be used in a three-drug combination treatment if no adequate glycemic control has been achieved with a two-drug combination (Garber A J, et al. Endocr Pract. 2019; 25: 69-100; Inzucchi S E, et al. Diabetes Care. 2015; 38: 140-9). The decision to use a sulfonylurea should take into account patient characteristics and potential adverse events that have been associated with sulfonylureas (Cordiner R L M, Pearson E R. Diabetes Obes Metab. 2019; 21: 761-771).

In one particularly preferred embodiment, the sulfonylurea is a Sur-1 receptor antagonist. Suitable Sur-1 receptor antagonists can be identified using known assays.

In one particularly preferred embodiment, the sulfonylurea is a SUR1-TRPM4 channel antagonist. Suitable SUR1-TRPM4 channel antagonists can be identified using known assays.

The invention also encompasses structural or functional analogues of the sulfonylureas, particularly those that are modified so as to extend the half-life of the agent, for example, conjugates of sulfonylureas.

In one preferred embodiment, the sulfonylurea is selected from glibenclamide (glyburide), glibornuride, gliclazide, glipizide, glimepiride, gliquidone, glisoxepide and glyclopyramide.

In one highly preferred embodiment, the sulfonylurea is selected from glibenclamide, and structural and functional analogues thereof.

Preferably, the sulfonylurea is selected from acylhydrazone, sulfonamide and sulfonylthiourea derivatives of glibenclamide, glimepiride, glipizide and gliclazide.

In one preferred embodiment, the sulfonylurea is glimepiride, which has the structure shown below:

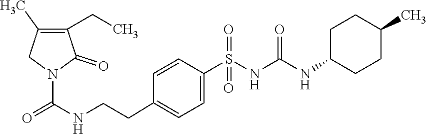

In one preferred embodiment, the sulfonylurea is gliclazide, which has the structure shown below:

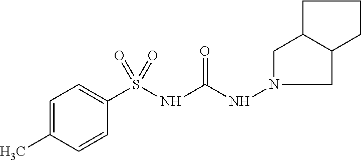

In one preferred embodiment, the sulfonylurea is glipizide, which has the structure shown below:

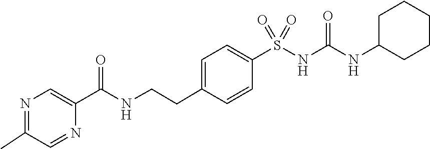 [SMILES: Cc1cnc(C(=O)NCCc2ccc(S(=O)(=O)NC(=O)NC3CCCCC3)cc2)cn1]

In one particularly preferred embodiment, the sulfonylurea is glibenclamide.

Glibenclamide has systematic (IUPAC) name 5-chloro-N-[2-[4-(cyclohexylcarbamoyl-sulfamoyl)phenyl]ethyl]-2-methoxybenzamide (chemical formula C₂₃H₂₈ClN₃O₅S) and its molecular weight is 494; it has the following chemical structure:

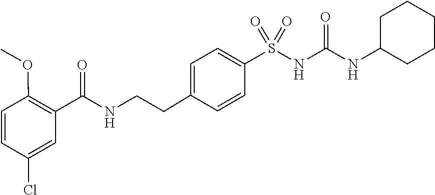 [SMILES: COc1ccc(Cl)cc1C(=O)NCCc1ccc(S(=O)(=O)NC(=O)NC2CCCCC2)cc1]

The invention also encompasses structural and functional analogues of the glibenclamide, particularly those that are modified so as to extend the half-life of the agent, for example, conjugates of glibenclamide.

Glibenclamide (also known as glyburide) is a sulfonylurea receptor-1 (Sur-1) receptor antagonist that is used as a hypoglycaemic agent to treat diabetes. Glibenclamide is being explored as a treatment to reduce oedema after brain injuries, such as ischemic stroke, traumatic brain injury, and subarachnoid haemorrhage, but the results so far are inconsistent (Wilkinson C M, et al. PLoS One. 2019; 14: e0215952; Xu F, et al. Brain Behav. 2019; 9(4): e01254; King Z A, et al. Drug Des Devel Ther. 2018; 12: 2539-2552). The present inventors investigated the role of glibenclamide as part of a combination therapy aiming to reduce reperfusion injury and leading potentially to a neuroprotective effect.

Glibenclamide is available as a generic and is sold in doses of 1.25, 2.5 and 5 mg under many brand names including Gliben-J, Daonil, Diabeta, Euglucon, Gilemal, Glidanil, Glybovin, Glynase, Maninil, Micronase and Semi-Daonil. Glibenclamide is used orally for the treatment of Type 2 diabetes, as a tablet formulation (for adults) or as an oral suspension (for children). The defined daily dose (DDD) of glibenclamide for the treatment of Type 2 diabetes is 7 mg for the micronized formulation, which has higher bioavailability and 10 mg for the conventional formulation. The defined daily dose (DDD) is the assumed average maintenance dose per day for a drug used for its main indication in adults, as defined in accordance with the WHO Collaborating Centre for Drug Statistics Methodology. The DDD is a unit of measurement and does not necessarily reflect the recommended or Prescribed Daily Dose. Therapeutic doses for individual patients and patient groups will often differ from the DDD as they will be based on individual characteristics (such as age, weight, ethnic differences, type and severity of disease) and pharmacokinetic considerations. The DDD value for glibenclamide is obtained from WHO Collaborating Centre for Drug Statistics Methodology (see https://www.whocc.no/atc_ddd_index/?code=A10BB01&showdescription=yes). The usual starting dose of glibenclamide (micronized formulation) as initial therapy is 2.5 to 5 mg daily and the usual maintenance dose is in the range of 1.25 to 20 mg daily, which may be given as a single dose or in divided doses, administered with breakfast or the first main meal (in accordance with the FDA label for Micronase® glyburide tablets). This corresponds to a maintenance dose of about 18 μg/kg to about 285 μg/kg for a 70 kg adult.

Several studies in animal models have demonstrated a protective role of glibenclamide in inflammation-associated injury including reduced adverse neuroinflammation and improved behavioral outcomes following central nervous system injury (Zhang G, et al. Mediators Inflamm. 2017; 2017: 3578702) or ischemic and hemorrhagic stroke (Caffes N, et al. Int J Mol Sci. 2015; 16: 4973-84). In a traumatic brain injury model in rats, glibenclamide was administered as loading dose of 10 μg/kg intraperitoneally followed by an infusion of 200 ng/hr for 7 days (Patel A D, et al. J Neuropathol Exp Neurol. 2010; 69: 1177-90), while in a mouse model of traumatic brain injury the dose of glibenclamide was 10 μg for three days after a controlled cortical impact injury (Xu Z M, et al. J Neurotrauma. 2017; 34: 925-933). In rodent models of brain ischemia and reperfusion injury, glibenclamide was shown to be effective at doses of 1 mg/kg administered 10 min before reperfusion (Abdallah D M, et al. Brain Res. 2011; 1385: 257-62). In rodent models of subarachnoid hemorrhage, glibenclamide was shown to be effective when administered as a loading dose of 10 μg/kg intraperitoneally followed by an infusion of 200 ng/hr for 24 hours (Simard, J. M, et al. Journal of Cerebral Blood Flow and Metabolism. 2009; 29; 317-330) or for one week (Tosun C, et al. Stroke. 2013; 44: 3522-8). Glibenclamide administered as a continuous infusion (75 ng/h) reduced cerebral edema, infarct volume and mortality by 50%, with the reduction in infarct volume being associated with cortical sparing, at 7 days post middle cerebral artery occlusion in a rat thromboembolic model of stroke (Simard J M, et al. Nat Med. 2006; 12: 433-40). Glibenclamide, administered at a dose of 10 μg either before or at 2 h after experimental intracerebral hemorrhage in mice, was shown to alleviate cerebral edema, disrupted BBB, and neurological deficit (Xu F, et al. Brain Behav. 2019; 9: e01254) and similar findings were obtained in another study (Jiang B, et al. Transl Stroke Res. 2017; 8: 183-193), but the widely-used glibenclamide dose which has been shown to be effective in other studies (10 μg/kg loading dose followed by 200 ng/h for up to 7 days) was shown to be not effective when the intracerebral hemorrhage was produced by intra-striatal injection of collagenase (Wilkinson C M, et al. PLoS One. 2019; 14(5): e0215952).

Glibenclamide was shown to exert beneficial effects in stroke patients also in some clinical trials. In the Glyburide Advantage in Malignant Edema and Stroke (GAMES) clinical trials in patients with large hemispheric infarctions, glyburide was administered intravenously (RP-1127) as a 0.13 mg bolus intravenous injection for the first 2 min, followed by an infusion of 0.16 mg/h for the first 6 h and then 0.11 mg/h for the remaining 66 h and revealed promising findings with regard to brain swelling (midline shift), MMP-9, functional outcomes and mortality (King Z A, et al. Drug Des Devel Ther. 2018; 12: 2539-2552). In an exploratory study with oral glibenclamide administration in patients with acute hemispheric infarction, treatment was shown to be safe, but it did not substantially improve 6-month functional outcome, although it was associated with lighter brain edema and a slight trend towards less severe disability and death was observed (Huang K, et al. Acta Neurol Scand. 2019 May 29). Retrospective analysis of data on diabetic patients who were not on a sulfonylurea to those who were during the days following acute ischemic strokes found a strong association between sulfonylurea treatment and improved survival, greater functional independence, lower NIH stroke scale scores, and less hemorrhagic transformation (Kunte H, et al. Ann Neurol. 2012; 72: 799-806).

The above preclinical and clinical findings are likely to be related to the upregulation of SUR1-TRPM4 channels after brain injuries such as ischemia (Woo S K, et al. J Biol Chem. 2013; 288: 3655-67; Mehta R I et al. J Neuropathol Exp Neurol. 2015; 74: 835-49).

Neuroprotective effects in animal models of ischemia and reperfusion injury have been reported also for other sulfonylureas, for example for gliclazide (Tan F, et al. Brain Res. 2014; 1560: 83-90), as well as protective effects in animal models of ischemia and reperfusion injury in other tissues, such as the myocardium for glimepiride (Nishida H et al. J Pharmacol Sci. 2009; 109: 251-6).

Some other drugs have insulin-secretagogue effects like the sulfonylureas; examples include the glinides (such as repaglinide, nateglinide and mitiglinide). Furthermore, other compounds, such as resveratrol, have been shown to bind to the sulfonylurea receptor (Hambrock A, et al. J Biol Chem. 2007; 282: 3347-56) and to have neuroprotective effects in stroke and traumatic CNS injury (Lopez M S, et al. Neurochem Int. 2015; 89: 75-82).

Studies by the Applicant, and described in more detail in the accompanying examples, have shown that administering a sulfonyl urea (e.g. glibenclamide) in combination with a second active which is either an aldosterone antagonist (e.g. potassium canrenoate) or an insulin modulator (e.g. exenatide) leads to a neuroprotective effect, even when the sulfonyl urea is administered at only very low doses.

### Insulin Modulator

In one embodiment, the combination of the invention comprises an insulin modulator, in addition to the above described sulfonylurea component.

As used herein the term “insulin modulator” refers to an agent that is capable of directly or indirectly increasing or decreasing the activity of insulin, which in turn may increase or decrease the insulin-mediated physiological response.

In one embodiment, the insulin modulator is selected from GLP-1 agonists, DPP-4 inhibitors, PPAR agonists, insulin and analogues thereof.

Examples of GLP-1 agonists include exenatide, lixisenatide, albiglutide, liraglutide, taspoglutide and dulaglutide (LY2189265) and pharmaceutically acceptable salts thereof.

Examples of DPP-4 inhibitors include sitagliptin, vildagliptin, saxagliptin, linagliptin anagliptin, teneligliptin, alogliptin, trelagliptin, gemigliptin, dutogliptin and omarigliptin (MK-3102) and pharmaceutically acceptable salts thereof.

Examples of PPAR agonists include clofibrate, gemfibrozil, ciprofibrate, bezafibrate, fenofibrate, saroglitazar, aleglitazar, muraglitazar and tesaglitazar and pharmaceutically acceptable salts thereof.

Examples of insulin analogues include insulin lispro, insulin aspart, insulin glulisine, insulin detemir, insulin degludec, insulin glargine and pharmaceutically acceptable salts thereof.

Accordingly, in one embodiment the insulin modulator is selected from exenatide, lixisenatide, albiglutide, liraglutide, taspoglutide, dulaglutide (LY2189265), sitagliptin, vildagliptin, saxagliptin, linagliptin anagliptin, teneligliptin, alogliptin, trelagliptin, gemigliptin, dutogliptin, omarigliptin (MK-3102), clofibrate, gemfibrozil, ciprofibrate, bezafibrate, fenofibrate, saroglitazar, aleglitazar, muraglitazar tesaglitazar, insulin lispro, insulin aspart, insulin glulisine, insulin detemir, insulin degludec, insulin glargine and pharmaceutically acceptable salts thereof.

In one embodiment, the insulin modulator is a GLP-1 agonist selected from exenatide, lixisenatide, albiglutide, liraglutide, taspoglutide, dulaglutide (LY2189265) and pharmaceutically acceptable salts thereof. Preferably, the GLP-1 agonist is exenatide.

### Exenatide

In one preferred embodiment, the insulin modulator is selected from exenatide and structural and functional analogues thereof, and pharmaceutically acceptable salts thereof.

In one preferred embodiment, the exenatide is in the form of a pharmaceutically acceptable salt, more preferably, exenatide acetate. In another preferred embodiment, the exenatide is in free base form.

As used herein, the term “exenatide” refers to a 39-mer peptide of the following sequence:

H-His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂

Exenatide (synonym is exendin 4) is originally isolated from the saliva of the Gila monster, Heloderma suspectum, by Eng in 1992. It is an insulin secretagogue with glucoregulatory effects similar to the human peptide glucagon-like peptide-1 (GLP-1).

Exenatide mimics human glucagons-like peptide 1 (GLP-1), a gut incretin hormone that is release in response to nutrient intake (Goke et al., J. Biol. Chem., 1993, 268: 19650-19655). It exerts insulinotropic and insulinomimetic properties via the GLP-1 receptor. GLP-1 receptor is widely expressed in many organs, including heart and vascular endothelium (Bullock et al., Endocrinology, 1996, 137: 2968-2978; Nystrom et al., Am J Physiol Endocrinol Metab, 2004, 287: E1209-E1215). Currently, exenatide is approved as an anti-diabetic drug for the treatment of patients with diabetes mellitus type 2. The recommended dose in this indication is initially 5 μg (μg) twice daily, increasing to 10 μg twice daily after 1 month based on clinical response.

GLP-1 is ineffective as a therapeutic agent as it has a very short circulating half-life (less than 2 minutes) due to rapid degradation by dipeptidyl peptidase-4. Exenatide is 50% homologous to GLP-1, but has a 2.4 hour half-life in humans as the dipeptidyl peprtidase-4 cleavage site is absent.

Exenatide enhances glucose-dependent insulin secretion by the pancreatic beta-cell, suppresses inappropriately elevated glucagon secretion, and slows gastric emptying. Exenatide is extremely potent, having a minimum effective concentration of 50 μg/mL (12 pM) in humans. Current therapies with exenatide involve twice-daily injections (Byetta®). Also, a slow-release formulation (Bydureon®) has been approved for once-weekly injection.

As used herein a functional analogue of exenatide refers to a compound having a similar structure, but differing from it in a respect of certain aspects (e.g. it can differ in one or more atoms, functional groups, amino acids residues, or substructures, which are replaced with others). Functional analogues display similar pharmacological properties and may be structurally related.

In one embodiment, the structural or functional analogue of exenatide is a form of exenatide that is modified so as to extend the half-life, for example, conjugates of exenatide.

In one preferred embodiment, the structural or functional analogue of exenatide is PEGylated exenatide. For example, in one preferred embodiment, the structural or functional analogue is exenatide mono-PEGylated with 40 kDa PEG. PEGylated exenatide can be prepared by methods known in the art. By way of example, PEGylated forms of exenatide are described in WO 2013/059323 (Prolynx LLC), the contents of which are hereby incorporated by reference. Exenatide can also be conjugated to other molecules, e.g. proteins.

In one particularly preferred embodiment, the structural or functional analogue of exenatide is an extended release form, for example, that marketed under the tradename Bydureon®. In another preferred embodiment, the structural or functional analogue of exenatide is in the form of multilayer nanoparticles for sustained delivery, for example, as described in Kim J Y et al, Biomaterials, 2013; 34:8444-9, the contents of which are hereby incorporated by reference.

In another particularly preferred embodiment, the exenatide is in an injectable form such as that marketed under the tradename Byetta®.

Functional analogues of exenatide include GLP receptor agonists. Suitable functional analogues of exenatide include lixisenatide, albiglutide, liraglutide, taspoglutide and dulaglutide (LY2189265).

In one embodiment, functional analogues of exenatide include exenatide modified wherein one or more amino acid residues has been exchanged for another amino acid residue and/or wherein one or more amino acid residues have been deleted and/or wherein one or more amino acid residues have been added and/or inserted.

In one embodiment, a functional exenatide analogue comprises less than 10 amino acid modifications (substitutions, deletions, additions (including insertions) and any combination thereof) relative to exenatide, alternatively less than 9, 8, 7, 6, 5, 4, 3 or 2 modifications relative to exenatide.

In one embodiment, a functional exenatide analogue comprises 10 amino acid modifications (substitutions, deletions, additions (including insertions) and any combination thereof) relative to exenatide, alternatively 9, 8, 7, 6, 5, 4, 3, 2 or 1 modifications relative to exenatide.

Structural and functional analogues of exenatide also include salts, isomers, enantiomers, solvates, polymorphs, prodrugs and metabolites thereof.

### Aldosterone Antagonist

In one embodiment, the combination of the invention comprises an aldosterone antagonist in addition to the above described sulfonylurea component.

Acute myocardial infarction and its subsequent hemodynamic changes lead to complex neurohormonal activation. The renin-angiotensin-aldosterone pathway is one cornerstone of such neurohormonal activation. Aldosterone, which is at its highest levels at presentation after acute myocardial infarction, is reported to promote a broad spectrum of deleterious cardiovascular effects including acute endothelial dysfunction, inhibition of NO activity, increased endothelial oxidative stress, increased vascular tone, inhibition of tissue recapture of catecholamines, rapid occurrence of vascular smooth muscle cell and cardiac myocyte necrosis, collagen deposition in blood vessels, myocardial hypertrophy, and fibrosis (Struthers, Am Heart J, 2002, 144: S2-S7; Zannad and Radauceanu, Heart Fail Rev, 2005, 10: 71-78). Furthermore, it has been found to predict poor outcomes (Beygui et al, Circulation, 2006, 114: 2604-2610).

An aldosterone antagonist or an antimineralocorticoid, is a diuretic drug which antagonizes the action of aldosterone at mineralocorticoid receptors. This group of drugs is often used for the management of chronic heart failure. Members of this class are also used in the management of hyperaldosteronism (including Conn's syndrome) and female hirsutism (due to additional antiandrogen actions). Most antimineralocorticoids are steroidal spirolactones.

Antagonism of mineralocorticoid receptors inhibits sodium resorption in the collecting duct of the nephron in the kidneys. This interferes with sodium/potassium exchange, reducing urinary potassium excretion and weakly increasing water excretion (diuresis). In congestive heart failure, aldosterone antagonists are used in addition to other drugs for additive diuretic effect, which reduces edema and the cardiac workload.

Current guidelines recommend the use of mineralocorticoid receptor antagonists, in patients presenting with heart failure post myocardial infarction, based on the results of the EPHESUS trial.

Several studies in animal models of acute myocardial infarction and in the clinic have shown the benefit of aldosterone blockade in the prevention of reperfusion injury and improving heart function in STEMI patient. There are indications in the literature that mineralocorticoid receptor antagonists may have beneficial effects in the cerebral vasculature and during stroke (Dinh Q N, et al. Neural Regen Res. 2016; 11:1230-1).

Examples of aldosterone antagonists include spironolactone (the first and most widely used member of this class), eplerenone (much more selective than spironolactone on target, but somewhat less potent and efficacious), canrenone and potassium canrenoate, finerenone (non-steroidal and more potent and selective than either eplerenone or spironolactone) and prorenone. Some drugs also have antimineralocorticoid effects secondary to their main mechanism of actions. Examples include progesterone, drospirenone, gestodene, and benidipine.

In one particularly preferred embodiment, the aldosterone antagonist is potassium canrenoate.

The invention also encompasses structural and functional analogues of aldosterone antagonists, particularly those that are modified so as to extend the half life of the agent, for example, conjugates of aldosterone antagonists.

### Potassium Canrenoate

Potassium canrenoate or canrenoate potassium also known as the potassium salt of canrenoic acid, is an aldosterone antagonist of the spirolactone group. Like spironolactone, it is a prodrug, which is metabolized to canrenone in the body. Potassium canrenoate is typically given intravenously at doses ranging between 200 mg/day and 600 mg/day for the treatment of hyperaldosteronism or hypokaliaemia.

Potassium canrenoate has the systematic (IUPAC) name potassium 3-[(8R,9S,10R,13S,14S,17R)-17-hydroxy-10,13-dimethyl-3-oxo-2,8,9,11,12,14,15,16 octahydro-1H-cyclopenta[a]phenanthren-17-yl]propanoate, formula C₂₂H₂₉KO₄ and the following chemical structure:

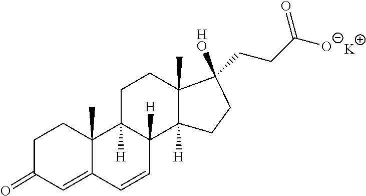

### Combinations

In one aspect, the present invention relates to a combination comprising:

(a) a sulfonylurea; and
(b) at least one of the following components: (i) an insulin modulator, and (ii) an aldosterone antagonist.

The preferred embodiments described below apply mutatis mutandis to other aspects of the invention, including methods, uses, products and compositions.

In one preferred embodiment, the combination comprises a sulfonylurea and an insulin modulator.

In one preferred embodiment, the combination consists of a sulfonylurea and an insulin modulator.

In another preferred embodiment, the combination comprises a sulfonylurea and an aldosterone antagonist.

In another preferred embodiment, the combination consists of a sulfonylurea and an aldosterone antagonist.

In another preferred embodiment, the combination comprises a sulfonylurea, an insulin modulator, and an aldosterone antagonist.

In another preferred embodiment, the combination consists of a sulfonylurea, an insulin modulator, and an aldosterone antagonist.

In one embodiment, the insulin modulator is defined according to any of the above-mentioned embodiments of an insulin modulator.

In one embodiment, the aldosterone antagonist is defined according to any of the above mentioned embodiments of an aldosterone antagonist.

In one embodiment, the sulfonylurea is defined according to any of the above mentioned embodiments of a sulfonylurea.

In one preferred embodiment, the present invention relates to a combination comprising:

(a) glibenclamide, or a structural or functional analogue thereof; and
(b) at least one of the following components: (i) exenatide, or a structural or functional analogue, or pharmaceutically acceptable salt thereof; and (ii) potassium canrenoate, or a structural or functional analogue thereof. Preferably, the combination comprises (b)(i) and (b)(ii).

In one preferred embodiment, the present invention relates to a combination comprising:

(a) at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
(b) at least one of the following components:
(i) at least one of exenatide, lixisenatide, albiglutide, liraglutide, taspoglutide and dulaglutide (LY2189265) or pharmaceutically acceptable salts thereof, and
(ii) at least one of potassium canrenoate, canrenone, spironolactone, eplerenone, finerenone and prorenone or pharmaceutically acceptable salts thereof, where applicable (e.g. pharmaceutically acceptable salts of canrenone, spironolactone, eplerenone, finerenone and prorenone).

In one embodiment, the present invention relates to a combination comprising:
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
at least one of exenatide, lixisenatide, albiglutide, liraglutide, taspoglutide and dulaglutide (LY2189265) or pharmaceutically acceptable salts thereof, and
at least one of potassium canrenoate, canrenone, spironolactone, eplerenone, finerenone and prorenone or pharmaceutically acceptable salts thereof, where applicable.

In one embodiment, the present invention relates to a combination comprising:
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
at least one of exenatide, lixisenatide, albiglutide, liraglutide, taspoglutide and dulaglutide (LY2189265) or pharmaceutically acceptable salts thereof.

In another embodiment, the present invention relates to a combination comprising:
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
at least one of potassium canrenoate, canrenone, spironolactone, eplerenone, finerenone and prorenone or pharmaceutically acceptable salts thereof where applicable.

In one embodiment, the present invention relates to a combination of, or comprising:
exenatide or a pharmaceutically acceptable salt thereof, and
at least one of glibenclamide, glibornuride, gliclazide, glipizide, glimepiride, gliquidone, glisoxepide, glyclopyramide, chlorpropamide, tolbutamide, acetohexamide, carbutamide, glycyclamide, tolhexamide, metahexamide, and tolazamide.

In one embodiment, the present invention relates to a combination of, or comprising:
exenatide or a pharmaceutically acceptable salt thereof, and
at least one of glibenclamide, glibornuride, gliclazide, glipizide, glimepiride, gliquidone, glisoxepide, and glyclopyramide.

In one embodiment, the present invention relates to a combination of, or comprising:
exenatide or a pharmaceutically acceptable salt thereof, and
glibenclamide.

In one embodiment, the present invention relates to a combination of, or comprising:
at least one of exenatide, lixisenatide, albiglutide, liraglutide, taspoglutide and dulaglutide (LY2189265) or a pharmaceutically acceptable salt thereof thereof, and
glibenclamide.

In one embodiment, the present invention relates to a combination of, or comprising:
potassium canrenoate, and
at least one of glibenclamide glibornuride, gliclazide, glipizide, glimepiride, gliquidone, glisoxepide, glyclopyramide, chlorpropamide, tolbutamide, acetohexamide, carbutamide, glycyclamide, tolhexamide, metahexamide, and tolazamide.

In one embodiment, the present invention relates to a combination of, or comprising:
potassium canrenoate, and
at least one of glibenclamide, glibornuride, gliclazide, glipizide, glimepiride, gliquidone, glisoxepide and glyclopyramide.

In one embodiment, the present invention relates to a combination of, or comprising:
at least one of potassium canrenoate, canrenone, spironolactone, eplerenone, finerenone and prorenone or pharmaceutically acceptable salts thereof where applicable, and
glibenclamide.

In one embodiment, the present invention relates to a combination of, or comprising:
potassium canrenoate; and
glibenclamide.

In one embodiment, the present invention relates to a combination comprising:
glibenclamide; and
at least one of exenatide, lixisenatide, albiglutide, liraglutide, taspoglutide and dulaglutide (LY2189265) or pharmaceutically acceptable salts thereof, and
at least one of potassium canrenoate, canrenone, spironolactone, eplerenone, finerenone and prorenone or pharmaceutically acceptable salts thereof, where applicable.

In one embodiment, the present invention relates to a combination comprising:
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
exenatide or pharmaceutically acceptable salts thereof, and
at least one of potassium canrenoate, canrenone, spironolactone, eplerenone, finerenone and prorenone or pharmaceutically acceptable salts thereof, where applicable.

In one embodiment, the present invention relates to a combination comprising:
at least one of glibenclamide, glibornuride, gliclazide, glipizide, glimepiride, gliquidone, glisoxepide glyclopyramide, chlorpropamide, tolbutamide, acetohexamide, carbutamide, glycyclamide, tolhexamide, metahexamide, and tolazamide; and
at least one of exenatide, lixisenatide, albiglutide, liraglutide, taspoglutide and dulaglutide (LY2189265) or pharmaceutically acceptable salts thereof, and
potassium canrenoate or canrenone.

In one embodiment, the present invention relates to a combination of, or comprising:
exenatide or a pharmaceutically acceptable salt thereof
potassium canrenoate; and
glibenclamide.

In one aspect of the invention, for each of the above embodiments, the combination consists of the sulfonyl urea and the aldosterone antagonist and/or insulin modulator, i.e. these are the only active agents present. In another (alternative) aspect, the combination further comprises one or more additional active agents as described hereinafter.

The effect of drug combinations is inherently unpredictable and there is often a propensity for one drug to partially or completely inhibit the effects of the other. The present invention demonstrates that a combination comprising a sulfonylurea such as glibenclamide or a structural or functional analogue thereof, and at least one of (i) an insulin modulator, such as exenatide or a structural or functional analogue or a pharmaceutically acceptable salt thereof, and (ii) an aldosterone antagonist, such as potassium canrenoate or a structural or functional analogue, when administered simultaneously, separately or sequentially, does not lead to any significant or dramatic adverse interaction between the two agents. The unexpected absence of any such antagonistic interaction is critical for clinical applications of the combination.

Moreover, preferred combinations according to the invention surprisingly demonstrate a potentiation of the effect of the individual components, such that the optimal doses for the agents is lower than the doses recommended in the approved indications for these agents, and/or also lower than the doses reported in the literature for reperfusion injury.

In one embodiment, the combinations of the active agents of the present invention produce an enhanced effect as compared to each drug administered alone.

By way of illustration, studies by the Applicant have shown that the preferred doses of glibenclamide which produce a synergistic effect in the context of the presently claimed combinations are significantly lower than the doses previously reported in the literature for blood glucose lowering (e.g. diabetes mellitus). In fact, the preferred doses of glibenclamide used in the presently claimed combinations are approximately ˜20 to 285-fold less than the recommended maintenance dose of glibenclamide (micronized formulation) for treating diabetes mellitus (for the micronized formulation of glibenclamide, the recommended maintenance daily dose is 1.25 to 20 mg, which corresponds to 18 μg/kg to 285 μg/kg for a 70 kg adult—contrast with the preferred doses of glibenclamide required in the presently claimed combination treatment, which can be as low as 1 μg/kg body weight). Advantageously, using glibenclamide in these preferred lower doses avoids any effect on blood glucose levels which could otherwise lead to adverse side effects. Studies by the Applicant have also shown that clinically effective doses of glibenclamide as a double or triple combination according to the invention with low doses of exenatide and/or potassium carbonate are also significantly lower than the doses of glibenclamide shown to be neuroprotective (continuous infusions of 0.16 or 0.11 mg/h, that is 3.84 mg or 2.64 mg daily) in clinical studies published in the literature (see King Z A et al).

Furthermore, in another embodiment, the combinations of the active agents of the present invention produce unexpected synergistic effects, for instance, in the treatment and/or prevention of reperfusion injury, particularly cerebral or myocardial reperfusion injury.

A combination of two or more drugs may lead to different types of drug interaction. A drug interaction is said to be additive when the combined effect of two drugs equals the sum of the effect of each agent given alone. A drug interaction is said to be synergistic if the combined effect of the two drugs exceeds the effects of each drug given alone (Goodman and Gilmans “The Pharmacological Basis of Therapeutics”, 12th Edition).

Combination therapy is an important treatment modality in many disease settings, including cardiovascular disease, cancer and infectious diseases. Recent scientific advances have increased the understanding of the pathophysiological processes that underlie these and other complex diseases. This increased understanding has provided further impetus to develop new therapeutic approaches using combinations of drugs directed at multiple therapeutic targets to improve treatment response, minimize development of resistance, or minimize adverse events. In settings in which combination therapy provides significant therapeutic advantages, there is growing interest in the development of new combinations of two or more drugs.

Advantageously, a synergistic combination may allow for lower doses of each component to be present, thereby decreasing the toxicity of therapy, whilst producing and/or maintaining the same therapeutic effect or an enhanced therapeutic effect. Thus, in a particularly preferred embodiment, each component of the combination is present in a sub-therapeutic amount. The term “sub-therapeutically effective amount” means an amount that is lower than that typically required to produce a therapeutic effect with respect to treatment with each agent alone.

In one embodiment, the present invention relates to a synergistic combination comprising a sulfonyl urea and an insulin modulator.

In another embodiment, the invention relates to a synergistic combination comprising a sulfonyl urea and an aldosterone antagonist.

In another embodiment, the invention relates to a synergistic combination comprising a sulfonyl urea, an insulin modulator and an aldosterone antagonist.

In one embodiment, the insulin modulator is defined according to any of the above mentioned embodiments of an insulin modulator.

In one embodiment, the aldosterone antagonist is defined according to any of the above mentioned embodiments of an aldosterone antagonist.

In one embodiment, the sulfonyl urea is defined according to any of the above mentioned embodiments of sulfonyl urea.

In one embodiment, the present invention relates to a synergistic combination comprising a sulfonylurea, and at least one of exenatide, lixisenatide, albiglutide, liraglutide, taspoglutide and dulaglutide (LY2189265) or a pharmaceutically acceptable salt thereof.

In one embodiment, the present invention relates to a synergistic combination comprising a sulfonylurea, and at least one of potassium canrenoate, canrenone, spironolactone, eplerenone, finerenone and prorenone or pharmaceutically acceptable salts thereof, where applicable.

In one embodiment, the present invention relates to a synergistic combination comprising:
at least one of glibenclamide, glibornuride, gliclazide, glipizide, glimepiride, gliquidone, glisoxepide, and glyclopyramide; and
at least one of exenatide, or a pharmaceutically acceptable salt thereof, and potassium canrenoate.

In one embodiment, the present invention relates to a synergistic combination comprising:
at least one of glibenclamide, glibornuride, gliclazide, glipizide, glimepiride, gliquidone, glisoxepide, and glyclopyramide;
at least one of exenatide, lixisenatide, albiglutide, liraglutide, taspoglutide and dulaglutide (LY2189265) or a pharmaceutically acceptable salt thereof; and
potassium canrenoate.

In one embodiment, the present invention relates to a synergistic combination comprising:
at least one of exenatide, lixisenatide, albiglutide, liraglutide, taspoglutide and dulaglutide (LY2189265) or a pharmaceutically acceptable salt thereof; and
glibenclamide.

In one embodiment, the present invention relates to a synergistic combination comprising:
exenatide or a pharmaceutically acceptable salt thereof; and
glibenclamide.

In one embodiment, the present invention relates to a synergistic combination comprising:
exenatide or a pharmaceutically acceptable salt thereof; and
at least one of glibenclamide, glibornuride, gliclazide, glipizide, glimepiride, gliquidone, glisoxepide, and glyclopyramide.

In one embodiment, the present invention relates to a synergistic combination comprising:
at least one of potassium canrenoate, canrenone, spironolactone, eplerenone, finerenone and prorenone or pharmaceutically acceptable salts thereof; and
glibenclamide.

In one embodiment, the present invention relates to a synergistic combination comprising:
potassium canrenoate; and
at least one of glibenclamide, glibornuride, gliclazide, glipizide, glimepiride, gliquidone, glisoxepide, and glyclopyramide.

In one embodiment, the present invention relates to a synergistic combination comprising potassium canrenoate and glibenclamide.

In one embodiment, the present invention relates to a synergistic combination comprising exenatide or a pharmaceutically acceptable salt thereof, potassium canrenoate and glibenclamide.

### Additional Active Pharmaceutical Ingredients

In one embodiment, the above described combinations comprise at least one further active pharmaceutical ingredient (API).

In one embodiment, the above described combinations may further comprise at least one further API selected from a beta blocker, a renin-angiotensin inhibitor, a statin (HMG-CoA reductase inhibitor), an inhibitor of platelet activation or aggregation, a phosphodiesterase-3 inhibitor, a calcium sensitizer, an antioxidant, and an anti-inflammatory agent.

Examples of beta-blockers include propranolol, metoprolol, bucindolol, carteolol, carvedilol, labetalol, nadolol, oxprenolol, penbutolol, pindolol, sotalol and timolol.

Renin-angiotensin inhibitors include angiotensin converting enzyme inhibitors, angiotensin AT, receptor inhibitors and renin inhibitors.

Examples of angiotensin converting enzyme inhibitors include captopril, zofenopril, enalapril, ramipril, quinapril, perindopril, lisinopril, benazepril, imidapril, trandolapril, cilazapril, and fosinopril.

Examples of angiotension AT, receptor antagonists include losartan, irbesartan, olmesartan, candesartan, valsartan, fimasartan and telmisartan.

Examples of renin inhibitors include remikiren and aliskiren.

Examples of calcium sensitizers include levosimendan and analogues thereof.

Examples of statins include atorvastatin, lovastatin, pravastatin, rosuvastatin and simvastatin.

Examples of platelet activation or aggregation inhibitors include prostacyclin (epoprostenol) and structural and functional analogues thereof (eg. treprostinil, iloprost), irreversible cyclooxygenase inhibitors (e.g. Aspirin, Triflusal), adenosine diphosphate (ADP) receptor inhibitors (e.g. Clopidogrel, Prasugrel, Ticagrelor, Ticlopidine), phosphodiesterase inhibitors (e.g. Cilostazol), protease-activated receptor-1 (PAR-1) antagonists (e.g. Vorapaxar), glycoprotein IIB/IIIA inhibitors (e.g. Abciximab, Eptifibatide, Tirofiban), adenosine reuptake inhibitors (e.g. Dipyridamole), and thromboxane inhibitors, including thromboxane synthase inhibitors and thromboxane receptor antagonists (e.g. Terutroban).

Examples of phosphodiesterase-3 (PDE-3) inhibitors include amrinone, milrinone, and analogues thereof.

Examples of antioxidants include ascorbic acid, lipoic acid, glutathione, melatonin and resveratrol.

Examples of anti-inflammatory agents include COX-2 inhibitors (e.g. celecoxib), glucocorticoids (e.g. hydrocortisone), and non-steroidal anti-inflammatory drugs (e.g. ibuprofen).

In one embodiment, the above combinations comprise at least one further API selected from propranolol, metoprolol, bucindolol, carteolol, carvedilol, labetalol, nadolol, oxprenolol, penbutolol, pindolol, sotalol, timolol, captopril, zofenopril, enalapril, ramipril, quinapril, perindopril, lisinopril, benazepril, imidapril, trandolapril, cilazapril, fosinopril, losartan, irbesartan, olmesartan, candesartan, valsartan, fimasartan, telmisartan, remikiren, aliskiren, melatonin and resveratrol.

In another embodiment, the above combinations comprise at least one further API selected from carvedilol, metoprolol, losartan, irbesartan, olmesartan, candesartan, valsartan, fimasartan telmisartan. captopril, zofenopril, enalapril, ramipril, quinapril, perindopril, lisinopril, benazepril, imidapril, trandolapril, cilazapril, fosinopril, remikiren aliskiren, melatonin and resveratrol.

In another embodiment, the above combinations comprise at least one further API selected from carvedilol, metoprolol, melatonin and resveratrol.

### Pharmaceutically Acceptable Salts

The active pharmaceutical agents of the present invention can be present as pharmaceutically acceptable salts.

Pharmaceutically acceptable salts of the agents of the invention include suitable acid addition or base salts thereof. A review of suitable pharmaceutical salts may be found in Berge et al., J Pharm Sci, 66, 1-19 (1977). Salts are formed, for example with strong inorganic acids such as mineral acids, e.g. sulphuric acid, phosphoric acid or hydrohalic acids (e.g. HCl, HBr); with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl- or aryl-sulfonic acids which are unsubstituted or substituted (for example, by a halogen) such as methane- or p-toluene sulfonic acid.

### Enantiomers/Tautomers

The invention also includes where appropriate all enantiomers and tautomers of the active pharmaceutical agents. The person skilled in the art will recognise compounds that possess optical properties (one or more chiral carbon atoms) or tautomeric characteristics. The corresponding enantiomers and/or tautomers may be isolated/prepared by methods known in the art.

### Stereo and Geometric Isomers

Some of the active pharmaceutical agents of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the active pharmaceutical agents or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as 2H, 3H, 13C, 14C, 15N, 17O, 18O, 31P, 32P, 35S, 18F and 36Cl, respectively. Certain isotopic variations of the agent and pharmaceutically acceptable salts thereof, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes solvate forms of the active pharmaceutical agents of the present invention. The terms used in the claims encompass these forms.

### Polymorphs

The invention furthermore relates to active pharmaceutical agents of the present invention in their various crystalline forms, polymorphic forms and (an)hydrous forms. It is well established within the pharmaceutical industry that chemical compounds may be isolated in any of such forms by slightly varying the method of purification and or isolation from the solvents used in the synthetic preparation of such compounds.

### Pharmaceutical Compositions

In another aspect, the present invention relates to a pharmaceutical composition comprising a combination according to the invention as described above and a pharmaceutically acceptable carrier, diluent or excipient.

In one aspect, the present invention relates to a pharmaceutical composition comprising:

(a) a sulfonylurea; and
(b) at least one of the following components: (i) an insulin modulator, and (ii) an aldosterone antagonist;
and a pharmaceutically acceptable carrier, diluent or excipient.

In one preferred embodiment, the present invention relates to a pharmaceutical composition comprising:

(a) glibenclamide or a structural or functional analogue thereof; and a pharmaceutically acceptable carrier, diluent or excipient; and
(b) at least one of the following components: (i) exenatide, or a structural or functional analogue thereof, or pharmaceutically acceptable salt thereof; and (ii) potassium canrenoate, or a structural or functional analogue;
and a pharmaceutically acceptable carrier, diluent or excipient.

In one aspect, the present invention relates to a pharmaceutical composition consisting of:

(a) a sulfonylurea; and
(b) at least one of the following components: (i) an insulin modulator, and (ii) an aldosterone antagonist;
and a pharmaceutically acceptable carrier, diluent or excipient.

In one preferred embodiment, the present invention relates to a pharmaceutical composition consisting of:

(a) glibenclamide or a structural or functional analogue thereof; and a pharmaceutically acceptable carrier, diluent or excipient; and
(b) at least one of the following components: (i) exenatide, or a structural or functional analogue thereof, or pharmaceutically acceptable salt thereof; and (ii) potassium canrenoate, or a structural or functional analogue;
and a pharmaceutically acceptable carrier, diluent or excipient.

Even though the compounds of the present invention (including their pharmaceutically acceptable salts) can be administered alone, they will generally be administered in admixture with a pharmaceutical carrier, excipient or diluent, particularly for human therapy. The pharmaceutical compositions may be for human or non-human animal usage in human and veterinary medicine respectively.

Examples of such suitable excipients for the various different forms of pharmaceutical compositions described herein may be found in the “Handbook of Pharmaceutical Excipients”, 2nd Edition, (1994), edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

The choice of pharmaceutical carrier, excipient or diluent can be selected with regard to the intended route of administration and standard pharmaceutical practice. Examples of routes of administration include parenteral (e.g., intravenous, intramuscular, intradermal, intraperitoneal or subcutaneous), oral, inhalation, transdermal (topical), intraocular, iontophoretic, and transmucosal administration.

In one embodiment, the pharmaceutical composition is for parenteral administration (e.g., intravenous, intraarterial, intrathecal, intramuscular, intradermal, intraperitoneal or subcutaneous). Preferably, the compositions are prepared from sterile or sterilisable solutions.

In another embodiment, the pharmaceutical composition is for intravenous, intramuscular, or subcutaneous administration.

In another embodiment, the pharmaceutical composition is for intravenous administration.

Solutions or suspension used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl-alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. The pH can be adjusted with acids or bases, such as hydrochloric acid or sodium hydroxide.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™. or phosphate buffered saline (PBS). In all cases, a composition for parenteral administration must be sterile and should be fluid to the extent that easy syringeability exists. It should be stable under the conditions of manufacture and storage and must be preserved against the contaminating action of microorganisms such as bacteria and fungi.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compounds into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof. The invention also encompasses liposomal and nanoparticulate formulations comprising the active agents. Such formulations, along with methods for their preparation, will be familiar to a person of ordinary skill in the art.

### Pharmaceutical Products

In another aspect, the present invention relates to a pharmaceutical product comprising:

(a) a sulfonylurea; and
(b) at least one of the following components: (i) an insulin modulator, and (ii) an aldosterone antagonist.

In one preferred embodiment, the present invention relates to a pharmaceutical product comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue.

In another aspect, the present invention relates to a pharmaceutical product consisting of:

(a) a sulfonylurea; and
(b) at least one of the following components: (i) an insulin modulator, and (ii) an aldosterone antagonist.

In one preferred embodiment, the present invention relates to a pharmaceutical product consisting of:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue.

In one preferred embodiment, each of the components of the pharmaceutical product is for separate administration.

In one embodiment, the pharmaceutical product is a kit of parts containing all necessary equipment (e.g., vials of drug, vials of diluent, syringes and needles) for a treatment course.

The components of the kit and pharmaceutical product are as defined above. In a preferred embodiment, each component of the kit or pharmaceutical product is admixed with one or more pharmaceutically acceptable diluents, excipients and/or carriers.

In one embodiment, the kit comprises separate containers for each active agent. Said containers may be ampoules, disposable syringes or multiple dose vials.

In another embodiment, the kit comprises a container which comprises a combined preparation of each active agent.

The kit may further comprise instructions for the treatment and/or prevention of reperfusion injury.

### Medical Uses

The present invention further relates to use of the above described combination, pharmaceutical product or pharmaceutical composition in treating various therapeutic disorders as detailed below, and methods of treatment relating to the same.

In one preferred embodiment, each of the pharmaceutically active components of the combination, pharmaceutical product or pharmaceutical composition is administered separately.

In one aspect, the present invention relates to a combination comprising:

(a) a sulfonylurea; and
(b) at least one of the following components: (i) an insulin modulator, and (ii) an aldosterone antagonist;
for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection, for example, against neurotoxic drugs.

In one preferred embodiment, the combination is for use in providing neuroprotection. Preferably, the combination is for use in providing neuroprotection against neurotoxic drugs.

In one preferred embodiment, the present invention relates to a combination comprising:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection against neurotoxic drugs.

In another aspect, the present invention relates to a pharmaceutical composition comprising a combination comprising:

(a) a sulfonylurea; and
(b) at least one of the following components: (i) an insulin modulator, and (ii) an aldosterone antagonist;
for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection against neurotoxic drugs.

In one preferred embodiment, the present invention relates to a pharmaceutical composition comprising a combination comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection against neurotoxic drugs.

In another aspect, the present invention relates to a pharmaceutical product comprising:

(a) a sulfonylurea; and
(b) at least one of the following components: (i) an insulin modulator, and (ii) an aldosterone antagonist;
for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection against neurotoxic drugs, wherein the components are for administration simultaneously, sequentially or separately.

In one preferred embodiment, the present invention relates to a pharmaceutical product comprising:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection against neurotoxic drugs, wherein the components are for administration simultaneously, sequentially or separately.

In another aspect, the present invention relates to use of:

(a) a sulfonylurea; and
(b) at least one of the following components: (i) an insulin modulator, and (ii) an aldosterone antagonist;
in the manufacture of a medicament for the treatment and/or prevention of one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection against neurotoxic drugs.

In another preferred embodiment, the present invention relates to use of:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
in the manufacture of a medicament for the treatment and/or prevention of one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection against neurotoxic drugs.

In one embodiment, the present invention relates to a combination comprising:

(a) a sulfonylurea; and
(b) at least one of the following components: (i) an insulin modulator, and (ii) an aldosterone antagonist;
for use in the treatment and/or prevention of reperfusion injury.

In one preferred embodiment, the present invention relates to a combination comprising:

(a) glibenclamide or a structural or functional analogue thereof; and
(b) at least two of the following components: (i) exenatide, or a structural or functional analogue thereof, or pharmaceutically acceptable salt thereof; (ii) potassium canrenoate, or a structural or functional analogue thereof;
for use in the treatment and/or prevention of reperfusion injury.

In another embodiment, the present invention relates to a pharmaceutical composition comprising:

(a) a sulfonylurea; and
(b) at least one of the following components: (i) an insulin modulator, and (ii) an aldosterone antagonist;
for use in the treatment and/or prevention of reperfusion injury.

In another preferred embodiment, the present invention relates to a pharmaceutical composition comprising a combination comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of reperfusion injury.

In another embodiment, the present invention relates to a pharmaceutical product comprising:

(a) a sulfonylurea; and
(b) at least one of the following components: (i) an insulin modulator, and (ii) an aldosterone antagonist;
for use in the treatment and/or prevention of reperfusion injury, wherein the components are for administration simultaneously, sequentially or separately.

In another preferred embodiment, the present invention relates to a pharmaceutical product comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of reperfusion injury, wherein the components are for administration simultaneously, sequentially or separately.

In another embodiment, the present invention relates to use of:

(a) a sulfonylurea; and
(b) at least one of the following components: (i) an insulin modulator, and (ii) an aldosterone antagonist;
in the manufacture of a medicament for the treatment and/or prevention of reperfusion injury.

In another preferred embodiment, the present invention relates to use of:

(a) glibenclamide or a structural or functional analogue thereof,
(b) at least one of the following components: (i) exenatide, or a structural or functional analogue thereof, or pharmaceutically acceptable salt thereof; and (ii) potassium canrenoate, or a structural or functional analogue thereof;
in the manufacture of a medicament for the treatment and/or prevention of reperfusion injury.

In one preferred embodiment, the present invention relates to a combination comprising:

(a) a sulfonylurea; and
(b) at least one of the following components: (i) an insulin modulator, and (ii) an aldosterone antagonist;
for use in the treatment and/or prevention of ischemia.

In one preferred embodiment, the present invention relates to a combination comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of ischemia.

In another embodiment, the present invention relates to a pharmaceutical composition comprising a combination comprising:

(a) a sulfonylurea; and
(b) at least one of the following components: (i) an insulin modulator, and (ii) an aldosterone antagonist;
for use in the treatment and/or prevention of ischemia.

In another preferred embodiment, the present invention relates to a pharmaceutical composition comprising a combination comprising:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for use in the treatment and/or prevention of ischemia.

In another embodiment, the present invention relates to a pharmaceutical product comprising:

(a) a sulfonylurea; and
(b) at least one of the following components: (i) an insulin modulator, and (ii) an aldosterone antagonist;
for use in the treatment and/or prevention of ischemia, wherein the components are for administration simultaneously, sequentially or separately.

In another preferred embodiment, the present invention relates to a pharmaceutical product comprising:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for use in the treatment and/or prevention of ischemia, wherein the components are for administration simultaneously, sequentially or separately.

In another embodiment, the present invention relates to use of:

(a) a sulfonylurea; and
(b) at least one of the following components: (i) an insulin modulator, and (ii) an aldosterone antagonist;
in the manufacture of a medicament for the treatment and/or prevention of ischemia.

In another preferred embodiment, the present invention relates to use of:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
in the manufacture of a medicament for the treatment and/or prevention of ischemia.

In one preferred embodiment, the present invention relates to a combination comprising:

(a) a sulfonylurea; and
(b) at least one of the following components: (i) an insulin modulator, and (ii) an aldosterone antagonist;
for use in the treatment and/or prevention of stroke.

In one preferred embodiment, the present invention relates to a combination comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of stroke.

In another embodiment, the present invention relates to a pharmaceutical composition comprising a combination comprising:

(a) a sulfonylurea; and
(b) at least one of the following components: (i) an insulin modulator, and (ii) an aldosterone antagonist;
for use in the treatment and/or prevention of stroke.

In another preferred embodiment, the present invention relates to a pharmaceutical composition comprising a combination comprising:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for use in the treatment and/or prevention of stroke.

In another embodiment, the present invention relates to a pharmaceutical product comprising:

(a) a sulfonylurea; and
(b) at least one of the following components: (i) an insulin modulator, and (ii) an aldosterone antagonist;
for use in the treatment and/or prevention of stroke, wherein the components are for administration simultaneously, sequentially or separately.

In another preferred embodiment, the present invention relates to a pharmaceutical product comprising:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for use in the treatment and/or prevention of stroke, wherein the components are for administration simultaneously, sequentially or separately.

In another embodiment, the present invention relates to use of:

(a) a sulfonylurea; and
(b) at least one of the following components: (i) an insulin modulator, and (ii) an aldosterone antagonist;
in the manufacture of a medicament for the treatment and/or prevention of stroke.

In another preferred embodiment, the present invention relates to use of:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
in the manufacture of a medicament for the treatment and/or prevention of stroke.

In one preferred embodiment, the stroke is a haemorrhagic stroke.

In another preferred embodiment, the stroke is ischemic stroke.

As used herein, the term “reperfusion injury” refers to the damage to tissue caused when blood supply returns to the tissue after a period of ischemia. The absence of oxygen and nutrients from blood creates a condition in which the restoration of circulation results in inflammation, mitochondrial dysfunction and oxidative damage through the induction of oxidative stress rather than restoration of normal function. Reperfusion injury can occur after a spontaneously occurring event, e.g., arterial blockage, or a planned event, e.g., any of a number of surgical interventions. Myocardial reperfusion injury can occur, for example, after myocardial infarction or as a result of heart transplantation. Cerebral reperfusion injury can occur, for example, after ischemic stroke or as a result of neonatal asphyxia.

In one embodiment, the ischemia and/or reperfusion injury is ischemia and/or reperfusion injury of the brain, heart, lung, kidney, or other organ/tissue susceptible to ischemia and/or reperfusion injury.

In one embodiment, the ischemia and/or reperfusion injury is ischemia and/or reperfusion injury of the brain, preferably cerebral ischemia and/or cerebral reperfusion injury.

In one embodiment, the ischemia and/or reperfusion injury is ischemia and/or reperfusion injury of the heart, preferably myocardial ischemia and/or myocardial reperfusion injury.

In one embodiment, the present invention relates to a combination comprising:

(a) a sulfonylurea; and
(b) at least one of the following components: (i) an insulin modulator, and (ii) an aldosterone antagonist;
for use in the treatment and/or prevention of a neurodegenerative disorder.

In one preferred embodiment, the present invention relates to a combination comprising:

(a) glibenclamide or a structural or functional analogue thereof; and
(b) at least two of the following components: (i) exenatide, or a structural or functional analogue thereof, or pharmaceutically acceptable salt thereof; (ii) potassium canrenoate, or a structural or functional analogue thereof;
for use in the treatment and/or prevention of a neurodegenerative disorder.

In another embodiment, the present invention relates to a pharmaceutical composition comprising:

(a) a sulfonylurea; and
(b) at least one of the following components: (i) an insulin modulator, and (ii) an aldosterone antagonist;
for use in the treatment and/or prevention of a neurodegenerative disorder.

In another preferred embodiment, the present invention relates to a pharmaceutical composition comprising a combination comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of a neurodegenerative disorder.

In another embodiment, the present invention relates to a pharmaceutical product comprising:

(a) a sulfonylurea; and
(b) at least one of the following components: (i) an insulin modulator, and (ii) an aldosterone antagonist;
for use in the treatment and/or prevention of a neurodegenerative disorder, wherein the components are for administration simultaneously, sequentially or separately.

In another preferred embodiment, the present invention relates to a pharmaceutical product comprising:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for use in the treatment and/or prevention of a neurodegenerative disorder, wherein the components are for administration simultaneously, sequentially or separately.

In another embodiment, the present invention relates to use of:

(a) a sulfonylurea; and
(b) at least one of the following components: (i) an insulin modulator, and (ii) an aldosterone antagonist;
in the manufacture of a medicament for the treatment and/or prevention of a neurodegenerative disorder.

In another preferred embodiment, the present invention relates to use of:

(a) glibenclamide or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
in the manufacture of a medicament for the treatment and/or prevention of a neurodegenerative disorder.

In one preferred embodiment, the neurodegenerative disorder is selected from Parkinson's disease, amyotrophic lateral sclerosis (ALS), Huntingdon's disease and Alzheimer's disease.

In one preferred embodiment, the neurodegenerative disorder is Parkinson's disease.

In one preferred embodiment, the neurodegenerative disorder is amyotrophic lateral sclerosis (ALS).

In one preferred embodiment, the neurodegenerative disorder is vascular dementia.

In one preferred embodiment, the neurodegenerative disorder is Alzheimer's disease.

The insulin modulator, the aldosterone antagonist and the sulfonylurea may be for administration simultaneously, sequentially or separately (as part of a dosing regimen).

Exenatide or structural or functional analogues thereof or pharmaceutically acceptable salts thereof, potassium canrenoate or structural or functional analogues thereof, and glibenclamide or structural or functional analogues or pharmaceutically acceptable salts thereof, may be for administration simultaneously, sequentially or separately (as part of a dosing regimen).

As used herein, “simultaneously” is used to mean that the two agents are administered concurrently.

As used herein, “sequentially” is used to mean that the active agents are not administered concurrently, but one after the other. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administrations of the components will vary depending on the exact nature of the components, the interaction there between, and their respective half-lives. In contrast to “sequentially”, “separately” is used herein to mean that the gap between administering one agent and the other is significant i.e. the first administered agent may no longer be present in the bloodstream in a therapeutically effective amount when the second agent is administered.

In one embodiment, the components of the combination are for simultaneous administration.

### Methods of Treatment

In another aspect, the present invention relates to a method of treating and/or preventing ischemia and/or reperfusion injury, said method comprising simultaneously, sequentially or separately administering to a subject:

(a) a sulfonylurea; and
(b) at least one of the following components: (i) an insulin modulator, and (ii) an aldosterone antagonist.

In another preferred embodiment, the present invention relates to a method of treating and/or preventing ischemia and/or reperfusion injury, said method comprising simultaneously, sequentially or separately administering to a subject in need thereof:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

In one embodiment, the present invention relates to a method of treating and/or preventing reperfusion injury, said method comprising simultaneously, sequentially or separately administering to a subject in need thereof:

(a) a sulfonylurea; and
(b) at least one of the following components: (i) an insulin modulator, and (ii) an aldosterone antagonist.

In one preferred embodiment, the present invention relates to a method of treating and/or preventing reperfusion injury, said method comprising simultaneously, sequentially or separately administering to a subject in need thereof:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

In another embodiment, the present invention relates to a method of treating and/or preventing ischemia, said method comprising simultaneously, sequentially or separately administering to a subject in need thereof:

(a) a sulfonylurea; and
(b) at least one of the following components: (i) an insulin modulator, and (ii) an aldosterone antagonist.

In another embodiment, the present invention relates to a method of treating and/or preventing ischemia, said method comprising simultaneously, sequentially or separately administering to a subject in need thereof:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

In one embodiment, the method relates to treating and/or preventing ischemia and/or reperfusion injury of the brain, heart, lung, kidney, or other organ/tissue susceptible to ischemia and/or reperfusion injury.

In one embodiment, the method relates to treating and/or preventing reperfusion injury of the brain, heart, lung, kidney, or other organ/tissue susceptible to reperfusion injury.

In one embodiment, the method relates to treating and/or preventing ischemia of the brain, heart, lung, kidney, or other organ/tissue susceptible to ischemia.

In another embodiment, the method relates to treating and/or preventing ischemia and/or reperfusion injury of the brain, preferably cerebral ischemia and/or cerebral reperfusion injury.

In another embodiment, the method relates to treating and/or preventing reperfusion injury of the brain, preferably cerebral reperfusion injury.

In another embodiment, the method relates to treating and/or preventing ischemia and/or reperfusion injury of the heart, preferably myocardial ischemia and/or myocardial reperfusion injury.

In another embodiment, the method relates to treating and/or preventing reperfusion injury of the heart, preferably myocardial reperfusion injury.

In one particularly preferred embodiment, the method relates to treating and/or preventing acute myocardial infarction. Acute myocardial infarction is one of the most common clinical indications of reperfusion injury.

In one particularly preferred embodiment, the method relates to treating and/or preventing stroke.

In one preferred embodiment, the stroke is a haemorrhagic stroke.

In one particularly preferred embodiment, the method relates to treating and/or preventing ischemic stroke. Ischemic stroke is one of the most common clinical indications of reperfusion injury.

In another embodiment, the method relates to treating and/or preventing neonatal asphyxia.

Neonatal asphyxia (or perinatal asphyxia) is the medical condition resulting from deprivation of oxygen to a newborn infant that lasts long enough during the birth process to cause physical harm, usually to the brain. The most common cause of neonatal asphyxia is a drop in maternal blood pressure or other interference to the blood flow to the infant's brain during delivery, for example, due to inadequate circulation or perfusion, impaired respiratory effort, or inadequate ventilation.

Neonatal asphyxia can cause hypoxic damage to most of the infant's organs (heart, lungs, liver, gut, kidneys), but brain damage is of most concern and perhaps the least likely to quickly or completely heal. In more pronounced cases, an infant will survive, but with damage to the brain manifested as either mental, such as developmental delay or intellectual disability, or physical, such as spasticity. An infant suffering severe perinatal asphyxia usually has poor colour (cyanosis), perfusion, responsiveness, muscle tone, and respiratory effort. Extreme degrees of asphyxia can cause cardiac arrest and death. Neonatal asphyxia occurs in 2 to 10 per 1000 newborns that are born at term, and in higher instances for those that are born prematurely. WHO estimates that 4 million neonatal deaths occur yearly due to birth asphyxia, representing 38% of deaths of children under 5 years of age.

In another embodiment, the method relates to treating and/or preventing ischemia of the heart, preferably myocardial ischemia.

In another embodiment, the present invention relates to a method of treating and/or preventing stroke, said method comprising simultaneously, sequentially or separately administering to a subject in need thereof:

(a) a sulfonylurea; and
(b) at least one of the following components: (i) an insulin modulator, and (ii) an aldosterone antagonist.

In another embodiment, the present invention relates to a method of treating and/or preventing stroke, said method comprising simultaneously, sequentially or separately administering to a subject in need thereof:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

In another embodiment, the present invention relates to a method of treating and/or preventing a neurodegenerative disease, said method comprising simultaneously, sequentially or separately administering to a subject in need thereof:

(a) a sulfonylurea; and
(b) at least one of the following components: (i) an insulin modulator, and (ii) an aldosterone antagonist.

In another embodiment, the present invention relates to a method of treating and/or preventing a neurodegenerative disease, said method comprising simultaneously, sequentially or separately administering to a subject in need thereof:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

In another embodiment, the present invention relates to a method of providing neuroprotection said method comprising simultaneously, sequentially or separately administering to a subject in need thereof:

(a) a sulfonylurea; and
(b) at least one of the following components: (i) an insulin modulator, and (ii) an aldosterone antagonist.

In another embodiment, the present invention relates to a method of providing neuroprotection, said method comprising simultaneously, sequentially or separately administering to a subject in need thereof:

(a) glibenclamide or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof.

In one embodiment, the subject is a mammal, more preferably a human.

In one embodiment, the method comprises parenterally (e.g., intravenously, intramuscularly, intradermally, intraperitoneally or subcutaneously) administering the components to the subject. The pharmaceutically active components of the combination can be administered separately or as a combined formulation. Preferably, the pharmaceutically active components are administered separately.

In another embodiment, the method comprises intravenously, intramuscularly, or subcutaneously administering the components to the subject.

In another embodiment, the method comprises intravenously administering (the components to the subject.

Each component can be administered by the same or different route to the other components. Preferably, the components are administered by the same route.

In one embodiment, the combinations are administered to a donor subject and/or a recipient subject prior to and/or during and/or after heart transplant. For example, in some embodiments the combination may be administered to a first subject from which the heart organ will be removed for transplantation into a second subject. Additionally or alternatively, in some embodiments, the combination is administered to the extracted heart organ, prior to introduction into the second subject. Additionally or alternatively, in some embodiments, the combination therapy is administered to the second subject before, during and/or after heart transplant.

In one embodiment, the combinations are for administration to a subject with stroke. Stroke is when poor blood flow to the brain results in cell death. There are two main types of stroke: ischemic, due to lack of blood flow, and haemorrhagic, due to bleeding. They result in part of the brain not functioning properly. Signs and symptoms of a stroke may include an inability to move or feel on one side of the body, problems understanding or speaking, feeling like the world is spinning, or loss of vision to one side among others. An ischemic stroke is typically caused by blockage of a blood vessel. Ischemic stroke treatment includes surgery to open up (reperfusion) the arteries to the brain in those with problematic narrowing. An ischemic stroke, if detected within three to four and half hours, may be treatable with a medication that can break down the clot. In 2013, stroke was the second most frequent cause of death after coronary artery disease, accounting for 6.4 million deaths (12% of the total).

Ischemic stroke and acute myocardial infarction require emergency reperfusion in order to improve functional outcome (Patel and Saver, 2013, Stroke, 44: 94-98). Intravenous tissue-type plasminogen activator has long been the only reperfusion therapy with proven clinical benefit in patients with acute ischemic stroke. As it happens in acute myocardial infarction, endovascular methods restoring reperfusion in acute ischemic stroke may expose patients to increased ischemic/reperfusion injury, thereby hampering the benefit of recanalization by promoting haemorrhagic transformation and severe vasogenic oedema both considering as markers of reperfusion injury (Bai and Lyden. 2015; Int J Stroke, 10: 143-152). Experimental evidence indicates that brain ischemic reperfusion injury (as happens in myocardial reperfusion injury) may be attenuated by ischemic pre- and post-conditioning. Glibenclamide was shown to enhance the therapeutic benefits of early hypothermia after severe stroke in rats (Zhu S, et al. Aging Dis. 2018; 9: 685-695). In addition, in a clinically relevant rat model of stroke (middle cerebral artery occlusion) reperfusion was initiated 4.5 h later and concomitantly was administered recombinant tissue plasminogen activator followed by administration of glibenclamide (10 μg/kg IP loading dose plus 200 ng/h by constant subcutaneous infusion) beginning 4.5 h or 10 h after onset of ischemia; glibenclamide significantly reduced hemispheric swelling at 24 h and 48-h mortality and improved Garcia scores at 48 h suggesting that the treatment window for glibenclamide extends to 10 h after onset of ischemia. This finding is consistent with observations in retrospective clinical studies suggesting that the use of sulfonylureas are beneficial in the context of rt-PA-aided recanalization/reperfusion following acute ischemic stroke (Simard, J M, et al. Ann. N. Y. Acad. Sci. 2012; 1268: 95-107).

In one embodiment, the combinations are for administration to a subject with cardiogenic shock. Cardiogenic shock is a life-threatening medical condition resulting from an inadequate circulation of blood due to primary failure of the ventricles of the heart to function effectively. The condition occurs in 2-10% of patients hospitalized due to myocardial infarction and is the main cause of death among these patients (Holmes et al, 1995, J Am Coll Cardiol, 26: 668-674). More specifically, cardiogenic shock is the result of a complex process with failure of oxygen delivery, generalized ATP deficiency, and multi-organ dysfunction initiated by cardiac pump failure (Okuda, 2006, Shock, 25: 557-570). As this is a type of circulatory shock, there is insufficient perfusion of tissue to meet the demands for oxygen and nutrients. The condition involves increasingly more pervasive cell death from oxygen starvation (hypoxia) and nutrient starvation (e.g. low blood sugar). Because of this, it may lead to cardiac arrest (or circulatory arrest), which is an abrupt stopping of cardiac pump function (as well as stopped respiration and a loss of consciousness). Cardiogenic shock is defined by sustained low blood pressure with tissue hypoperfusion despite adequate left ventricular filling pressure. Signs of tissue hypoperfusion include low urine production (<30 mL/hour), cool extremities, and altered level of consciousness. Several large trials have demonstrated that coronary revascularization is the most important strategy to improve patient survival (Hochman et al, 1999, N Engl J Med, 341: 625-634). However, patients who develop cardiogenic shock despite acute revascularization have a poor prognosis, likely due to reperfusion injury and considered to be associated to the resulted infarct size. Indeed, hypothermia has shown to offer tissue protection in myocardial ischemia, and preclinical studies have shown beneficial results in reducing infarct size in experimentally induced myocardial infarction (Dae et al, 2002, Am J Physiol Heart Circ Physiol, 282: H1584-H1591). Accordingly, in a pig model mild therapeutic hypothermia reduced acute mortality in cardiogenic shock, and improved hemodynamic parameters (Gotberg et al, 2010, Resuscitation, 81: 1190-96)

In one embodiment, the combinations are for administration to a subject with cardiac arrest. Cardiac arrest is a sudden stop in effective blood flow due to the failure of the heart to contract effectively. The most common cause of cardiac arrest is coronary artery disease. Treatment for cardiac arrest is immediate cardiopulmonary resuscitation (CPR) and if a shockable rhythm is present, defibrillation. In the United States cardiac arrest outside of hospital occurs in about 13 per 10,000 people per year (326,000 cases). In hospital cardiac arrest occurs in an additional 209,000 (Kronic et al, Circulation, 2015, 132: S397-S413). In addition to providing high quality cardiopulmonary resuscitation, optimizing the management for post-cardiac arrest syndrome is critically important for improving the long term outcome for cardiac arrest patients. Within this syndrome (“post-cardiac arrest syndrome”) there are 3 major areas of emphasis: (1) post-cardiac arrest brain injury; (2) post-cardiac arrest myocardial dysfunction and reperfusion injury; and (3) systemic ischemia-reperfusion response. It is now clear that post-resuscitation care can affect long-term survival and the myocardial and neurological recovery and function of survivors (Kem, 2015, Circ J, 79: 1156-1163).

In one embodiment the subject is at risk of (or susceptible to) vessel occlusion injury or cardiac ischemia-reperfusion injury.

In one embodiment, the combinations are for use in, or methods of, providing neuroprotection in a subject.

As used herein, the term “neuroprotection” refers to protecting a neural entity, including the brain, for example, by preventing, reducing or delaying brain damage that may lead to the death of the neurons and to neurodegenerative disorders such as Alzheimer's disease, Parkinson's disease or vascular dementia.

In one embodiment, the combinations are for use in, or methods of, providing neuroprotection in a subject against the neurotoxic effects of drugs. Examples of neurotoxic drugs are described by Gouzoulis-Mayfrank and Daumann (Dialogues Clin Neurosci. 2009; 11(3):305-17). Neurotoxic drugs include drugs of abuse (eg. 3,4-methylendioxymethamphetamine, methamphetamine and amphetamine), pesticides (eg. organic phosphorus-based pesticides), certain chemotherapies (eg. platinum), and dopamine.

In one embodiment, the claimed combinations are for use in, or methods of, providing cardioprotection in a subject against the cardiotoxic effects of drugs (e.g. anthracyclines). Examples of cardiotoxic drugs are described in Bovelli et al (Annals of Oncology 21 (Supplement 5): v277-v282, 2010).

As used herein, the term “cardioprotection” refers to protecting the heart, for example, by preventing, reducing or delaying myocardial injury. Cardiotoxic drugs include drugs associated with cardiac heart failure, drugs associated with ischaemia or thromboembolism, drugs associated with hypertension, drugs associated with other toxic effects such as tamponade and endomyocardial fibrosis, haemorrhagic myocarditis, bradyarrhythmias, Raynaud's phenomenon, autonomic neuropathy, QT prolongation or torsades de pointes, or pulmonary fibrosis. Examples of cardiotoxic drugs include anthracyclines/anthraquinolones, cyclophosphamide, Trastuzumab and other monoclonal antibody-based tyrosine kinase inhibitors, antimetabolites (fluorouracil, capecitabine), antimicrotubule agents (paclitaxel, docetaxel), cisplatin, thalidomide, bevacizumab, sunitinib, sorafenib, busulfan, paclitaxel, vinblastine, bleomycin, vincristine, arsenic trioxide, bleomycin and methotrexate.

In one embodiment, the components are administered simultaneously.

In one embodiment, the components are administered sequentially or separately.

For a three component combination, all three components can be administered simultaneously, or any two components can be administered simultaneously, with the third component administered separately or sequentially. Alternatively, all three components can be administered in any order separately or sequentially.

In one embodiment, the sulfonylurea is administered prior to sequentially or separately administering the insulin modulator.

In another embodiment, the insulin modulator is administered prior to sequentially or separately administering the sulfonylurea.

In one embodiment, the sulfonylurea is administered prior to sequentially or separately administering the aldosterone antagonist.

In one embodiment, the aldosterone antagonist is administered prior to sequentially or separately administering the sulfonylurea.

In one embodiment, the exenatide, or a structural or functional analogue, or pharmaceutically acceptable salt thereof; potassium canrenoate, or a structural or functional analogue thereof; and glibenclamide or a structural or functional analogue thereof, are administered sequentially or separately.

In one embodiment, the components are each administered in a therapeutically effective amount with respect to the individual components.

As used herein, the term “therapeutically effective amount” refers to a quantity sufficient to achieve a desired therapeutic and/or prophylactic effect, e.g., an amount which results in the prevention of, or a decrease in, ischemia and/or reperfusion injury or one or more symptoms associated with ischemia and/or reperfusion injury.

In the context of therapeutic or prophylactic applications, the amount of a composition administered to the subject will depend on the type and severity of the disease and on the characteristics of the individual, such as general health, age, sex, body, weight and tolerance to drugs. It will also depend on the degree severity and type of disease. The skilled artisan will be able to determine appropriate dosages depending on these and other factors. The composition can also be administered in combination with one or more additional therapeutic agents.

In one embodiment, the components are each administered in a sub-therapeutically effective amount with respect to the individual components.

In one embodiment, the components are administered prior to reperfusion the subject.

In one embodiment, the components are administered during reperfusion of the subject.

In one embodiment, the components are administered after reperfusion of the subject.

In one embodiment, the components are administered prior to and/or during and/or after reperfusion of the subject.

In some embodiments of the method, the subject is administered the sulfonylurea continuously before, during, and after reperfusion of the subject and is administered the insulin modulator as a bolus dose prior to reperfusion.

In some embodiments of the method, the subject is administered the insulin modulator continuously before, during, and after reperfusion of the subject and is administered the sulfonylurea as a bolus dose prior to reperfusion.

In some embodiments of the method, the subject is administered the sulfonylurea continuously before, during, and after reperfusion of the subject and is administered the aldosterone antagonist as a bolus dose prior to reperfusion.

In some embodiments of the method, the subject is administered the aldosterone antagonist continuously before, during, and after reperfusion of the subject and is administered the sulfonylurea as a bolus dose prior to reperfusion.

In some embodiments of the method, the subject is administered the components continuously before, during, and after reperfusion of the subject.

In some embodiments of the method, additional administration of one or more of the components may occur after reperfusion. Preferably, this repeat administration is carried out at least twice, more preferably from 2 to 100 times, or can be in the form of continuous infusion.

In some embodiments of the method, the subject is administered the components as a bolus dose prior to reperfusion.

In some embodiments of the method, the subject is administered the components as a bolus dose during reperfusion.

In some embodiments of the method, the subject is administered the components as a bolus dose after reperfusion.

As used herein “reperfusion” is the restoration of blood flow to any organ or tissue in which the flow of blood is decreased or blocked. For example, blood flow can be restored to any organ or tissue affected by ischemia or hypoxia. The restoration of blood flow (reperfusion) can occur by any method known to those in the art. For instance, reperfusion of ischemic cardiac tissues may arise from revascularization.

In one embodiment, reperfusion is achieved via a revascularization procedure. In one embodiment, the revascularization procedure is selected from the group consisting of: percutaneous coronary intervention; balloon angioplasty; insertion of a bypass graft; insertion of a stent; directional coronary atherectomy; treatment with a one or more thrombolytic agent(s); and removal of an occlusion.

In one embodiment, the one or more thrombolytic agents are selected from the group consisting of: tissue plasminogen activator; urokinase; prourokinase; streptokinase; acylated form of plasminogen; acylated form of plasmin; and acylated streptokinase-plasminogen complex.

### Dosage

A person of ordinary skill in the art can easily determine an appropriate dose of one of the instant compositions to administer to a subject without undue experimentation.

Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In one highly preferred embodiment of the invention, the dose of the insulin modulator (e.g. exenatide) in the combination is generally lower than the dose typically used in monotherapy in the context of its currently approved therapies, and/or lower than the general doses reported in the reperfusion injury literature.

In one highly preferred embodiment of the invention, the dose of the aldosterone antagonist (e.g. potassium canrenoate) in the combination is generally lower than the dose typically used in monotherapy in the context of its currently approved therapies, and/or lower than the general doses reported in the reperfusion injury literature.

In one highly preferred embodiment of the invention, the dose of the sulfonylurea (e.g. glibenclamide) in the combination is generally lower than the dose typically used in monotherapy in the context of its currently approved therapies, and/or lower than the general doses reported in the reperfusion injury literature.

Each component of the claimed combination may be formulated in unit dosage form, i.e., in the form of discrete portions containing a unit dose, or a multiple or sub-unit of a unit dose. The dosages described herein are applicable to each of the above-described medical uses.

When used in the presently claimed combinations, the insulin modulator (e.g. exenatide) is preferably administered in a dose of from about 0.001 to about 1.5 μg/kg, more preferably from about 0.005 to about 0.15 μg/kg. In one preferred embodiment, the insulin modulator (e.g. exenatide) is preferably administered in a dose of from about 0.01 to about 1.5 μg/kg, more preferably from about 0.05 to about 1.5 μg/kg. As used herein the insulin modulator dosages are μg/kg body weight (μg=microgram).

In one preferred embodiment, the insulin modulator (e.g. exenatide) is preferably administered in a dose of from about 0.01 to about 0.5 μg/kg, more preferably from about 0.02 to about 0.5 μg/kg, or from about 0.03 to about 0.5 μg/kg, or from about 0.04 to about 0.5 μg/kg, or from about 0.05 to about 0.5 μg/kg, or from about 0.05 to about 0.2 μg/kg, or from about 0.05 to about 0.15 μg/kg.

In one preferred embodiment, the insulin modulator (e.g. exenatide) is preferably administered in a dose of from about 0.01 to about 0.1 μg/kg, more preferably from about 0.02 to about 0.08 μg/kg, or from about 0.03 to about 0.07 μg/kg, or from about 0.04 to about 0.06 μg/kg, or in a dose of about 0.05 μg/kg.

When used in the presently claimed combinations, the aldosterone antagonist (e.g. potassium canrenoate) is preferably administered in a dose of from about 0.03 to about 10 mg/kg, or about 0.1 to about 10 mg/kg or about 0.3 to about 5 mg/kg, or from about 1 to about 10 mg/kg, or from about 1 to about 5 mg/kg, or from about 1 to about 3 mg/kg. As used herein the aldosterone antagonist dosages are in mg/kg body weight.

In one preferred embodiment, the aldosterone antagonist (e.g. potassium canrenoate) is preferably administered in a dose of from about 0.1 to about 3 mg/kg or from about 0.2 to about 2 mg/kg, or from about 0.3 to about 1.5 mg/kg, or from about 0.3 to about 1 mg/kg.

In one preferred embodiment, the aldosterone antagonist (e.g. potassium canrenoate) is preferably administered in a dose of from about 0.1 to about 0.5 mg/kg or from about 0.2 to about 0.5 mg/kg, more preferably, from about 0.2 to about 0.4 mg/kg, even more preferably, about 0.3 to 0.4 mg/kg.

When used in the presently claimed combinations, the sulfonylurea (e.g. glibenclamide) is preferably administered in a dose of from about 0.001 to about 30 μg/kg, more preferably from about 0.01 to about 5 μg/kg, even more preferably from about 0.01 to about 2 μg/kg. As used herein the sulfonylurea dosages are in μg/kg body weight.

In one preferred embodiment, the sulfonylurea (e.g. glibenclamide) is preferably administered in a dose of from about 0.5 to about 20 μg/kg, or from about 0.5 to about 15 μg/kg, or from about 0.5 to about 10 μg/kg, or from about 1 to about 10 μg/kg.

In one preferred embodiment, the sulfonylurea (e.g. glibenclamide) is preferably administered in a dose of from about 0.5 to about 8 μg/kg, or from about 0.5 to about 7 μg/kg, or from about 0.5 to about 6 μg/kg, or from about 0.5 to about 5 μg/kg. In one preferred embodiment, the sulfonylurea (e.g. glibenclamide) is preferably administered in a dose of from about 0.5 to about 3 μg/kg, or from about 0.5 to about 2 μg/kg, or from about 0.5 to about 1.5 μg/kg, or from about 0.8 to about 1.2 μg/kg, or at about 1 μg/kg.

In one highly preferred embodiment, the combination is a fixed dose combination comprising predetermined dosages of the respective pharmaceutically active components e.g. to allow administration to the subject of the above described dosages, for example, about 0.005 to about 0.15 μg/kg exenatide and from about 0.001 to about 30 μg/kg glibenclamide.

Preferably, the fixed dose combination comprises predetermined dosages of the respective pharmaceutically active components to allow administration to the subject of the following doses.

In one highly preferred embodiment, the combination is a fixed dose combination comprising about 0.01 to about 0.5 μg/kg exenatide and from about 0.5 to about 20 μg/kg glibenclamide.

In one highly preferred embodiment, the combination is a fixed dose combination comprising about 0.01 to about 0.1 μg/kg exenatide and from about 0.5 to about 8 μg/kg glibenclamide.

In one highly preferred embodiment, the combination is a fixed dose combination comprising about 0.01 to about 0.1 μg/kg exenatide and from about 0.5 to about 1.5 μg/kg glibenclamide.

In one highly preferred embodiment, the combination is a fixed dose combination comprising predetermined dosages of the respective components e.g. about 0.03 to about 10 mg/kg potassium canrenoate and from about 0.001 to about 30 μg/kg glibenclamide.

In one highly preferred embodiment, the combination is a fixed dose combination comprising about 0.1 to about 3 mg/kg potassium canrenoate and from about 0.5 to about 20 μg/kg glibenclamide.

In one highly preferred embodiment, the combination is a fixed dose combination comprising about 0.1 to about 0.5 mg/kg potassium canrenoate and from about 0.5 to about 8 μg/kg glibenclamide.

In one highly preferred embodiment, the combination is a fixed dose combination comprising about 0.1 to about 0.5 mg/kg potassium canrenoate and from about 0.5 to about 1.5 μg/kg glibenclamide.

In one highly preferred embodiment, the combination is a fixed dose combination comprising predetermined dosages of the respective components e.g. about 0.03 to about 10 mg/kg potassium canrenoate, from about 0.001 to about 30 μg/kg glibenclamide and from about 0.005 to about 0.15 μg/kg exenatide.

In one highly preferred embodiment, the combination is a fixed dose combination comprising about 0.1 to about 3 mg/kg potassium canrenoate, from about 0.5 to about 20 μg/kg glibenclamide, and from about 0.01 to about 0.5 μg/kg exenatide.

In one highly preferred embodiment, the combination is a fixed dose combination comprising about 0.1 to about 0.5 mg/kg potassium canrenoate, from about 0.5 to about 8 μg/kg glibenclamide, and from about 0.01 to about 0.1 μg/kg exenatide.

In one highly preferred embodiment, the combination is a fixed dose combination comprising about 0.1 to about 0.5 mg/kg potassium canrenoate, from about 0.5 to about 1.5 μg/kg glibenclamide, and from about 0.01 to about 0.1 μg/kg exenatide.

In one highly preferred embodiment, the combination is a fixed dose combination comprising about 0.05 μg/kg exenatide and 1 μg/kg glibenclamide.

In one highly preferred embodiment, the combination is a fixed dose combination comprising about 0.33 mg/kg potassium canrenoate and about 1 μg/kg glibenclamide.

In one highly preferred embodiment, the combination is a fixed dose combination comprising about 0.05 μg/kg exenatide, about 0.33 mg/kg potassium canrenoate, and about 1 μg/kg glibenclamide.

### Non-Therapeutic Use

In another aspect, the present invention relates to use of a combination comprising:

(a) a sulfonylurea; and
(b) at least one of the following components: (i) an insulin modulator, and (ii) an aldosterone antagonist;
for treating and/or preventing ischemia and/or reperfusion injury in an ex vivo organ prior to or during transplantation.

In another preferred embodiment, the present invention relates to use of a combination comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for treating and/or preventing ischemia and/or reperfusion injury in an ex vivo organ prior to or during transplantation.

In one embodiment, the present invention relates to use of a combination comprising:

(a) a sulfonylurea; and
(b) at least one of the following components: (i) an insulin modulator, and (ii) an aldosterone antagonist;
for treating and/or preventing reperfusion injury in an ex vivo organ prior to or during transplantation.

In one preferred embodiment, the present invention relates to use of a combination comprising:

(a) glibenclamide, or a structural or functional analogue thereof; and
(b) at least one of the following components: (i) exenatide, or a structural or functional analogue thereof, or pharmaceutically acceptable salt thereof; (ii) potassium canrenoate, or a structural or functional analogue thereof;
for treating and/or preventing reperfusion injury in an ex vivo organ prior to or during transplantation.

In one embodiment, the present invention relates to use of a combination comprising:

(a) a sulfonylurea; and
(b) at least one of the following components: (i) an insulin modulator, and (ii) an aldosterone antagonist;
for treating and/or preventing ischemia in an ex vivo organ prior to or during transplantation.

In one preferred embodiment, the present invention relates to use of a combination comprising:

(a) glibenclamide, or a structural or functional analogue thereof; and
(b) at least one of the following components: (i) exenatide, or a structural or functional analogue thereof, or pharmaceutically acceptable salt thereof; and (ii) potassium canrenoate, or a structural or functional analogue thereof;
for treating and/or preventing ischemia in an ex vivo organ prior to or during transplantation.

An ex vivo (removed from the body) organ can be susceptible to reperfusion injury due to lack of blood flow. Therefore, the combination of the present invention can be used to prevent reperfusion injury in the removed organ. Preferably, the organ is a heart, liver or kidney, more preferably, a heart.

In some embodiments, the removed organ is placed in a standard buffered solution, such as those commonly used in the art, containing the combination of the invention. For example, a removed heart can be placed in a cardioplegic solution containing exenatide, potassium canrenoate and glibenclamide. The concentration of exenatide, potassium canrenoate and glibenclamide useful in the standard buffered solution can be easily determined by those skilled in the art. Such concentrations may be, for example, between about 0.1 nM to about 10 μM, preferably about 1 nM to about 10 μM.

The invention is further described with reference to the accompanying non-limiting examples, and the following figures wherein:

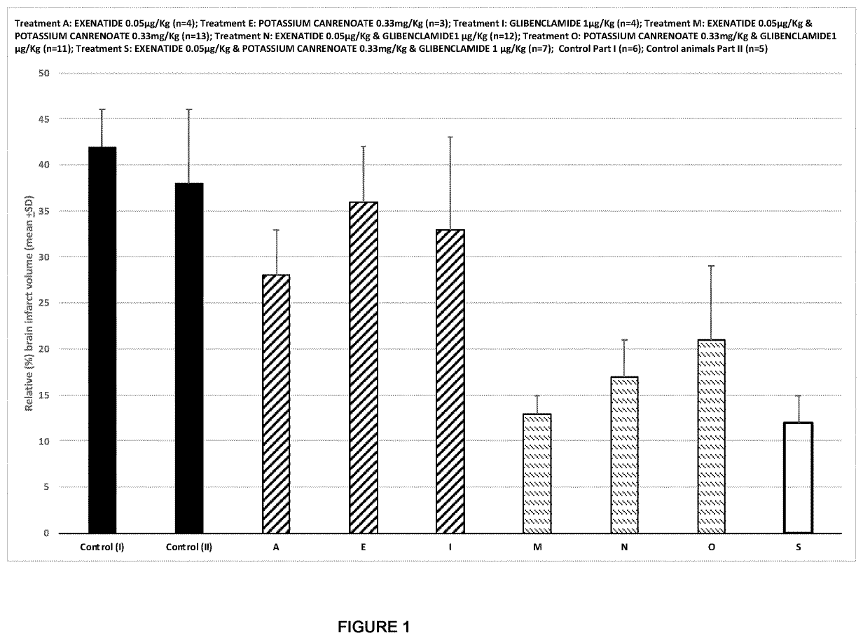

FIG. 1 shows relative brain infarct volume (%) on Day 7 following repeated administration (7 days) of the low dose triple combination (treatment S) versus that in control animals in Part I and Part II and in animals that received the corresponding monotherapies (treatments A, E, and I) and the corresponding double combinations (treatments M, N, and O) in a rat model of transient middle cerebral artery occlusion. Treatment A: Exenatide 0.05 μg/kg (n=4); Treatment E: Potassium canrenoate 0.33 mg/kg (n=3); Treatment I: Glibenclamide 1 μg/kg (n=4); Treatment M: Exenatide 0.05 μg/kg and Potassium canrenoate 0.33 mg/kg (n=13); Treatment N: Exenatide 0.05 μg/kg and Glibenclamide 1 μg/kg (n=12); Treatment O: Potassium canrenoate 0.33 mg/kg and Glibenclamide 1 μg/kg (n=11); Treatment S: Exenatide 0.05 μg/kg and Potassium canrenoate 0.33 mg/kg and Glibenclamide 1 μg/kg (n=7). Control Part I (n=6); Control animals Part II (n=5).

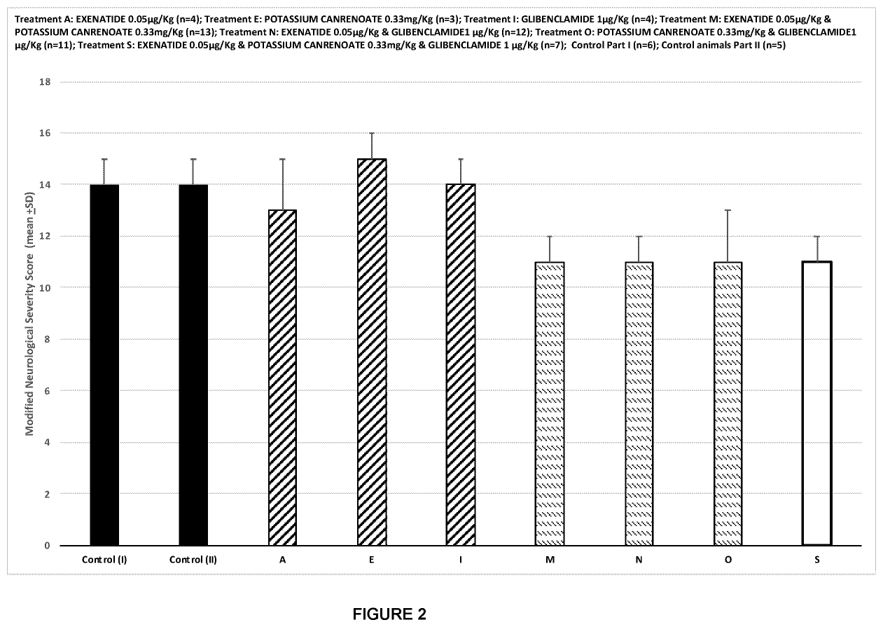

FIG. 2 shows the Modified Neurological Severity Score on Day 2 following repeated administration (7 days) of the low dose triple combination (treatment S) versus that in control animals in Part I and Part II and in animals that received the corresponding monotherapies (treatments A, E, and I) and the corresponding double combinations (treatments M, N, and O) in a rat model of transient middle cerebral artery occlusion. Treatment A: Exenatide 0.05 μg/kg (n=4); Treatment E: Potassium canrenoate 0.33 mg/kg (n=3); Treatment I: Glibenclamide 1 μg/kg (n=4); Treatment M: Exenatide 0.05 μg/kg and Potassium canrenoate 0.33 mg/kg (n=13); Treatment N: Exenatide 0.05 μg/kg and Glibenclamide 1 μg/kg (n=12); Treatment O: Potassium canrenoate 0.33 mg/kg and Glibenclamide 1 μg/kg (n=11); Treatment S: Exenatide 0.05 μg/kg and Potassium canrenoate 0.33 mg/kg and Glibenclamide 1 μg/kg (n=7). Control Part I (n=6); Control animals Part II (n=5).

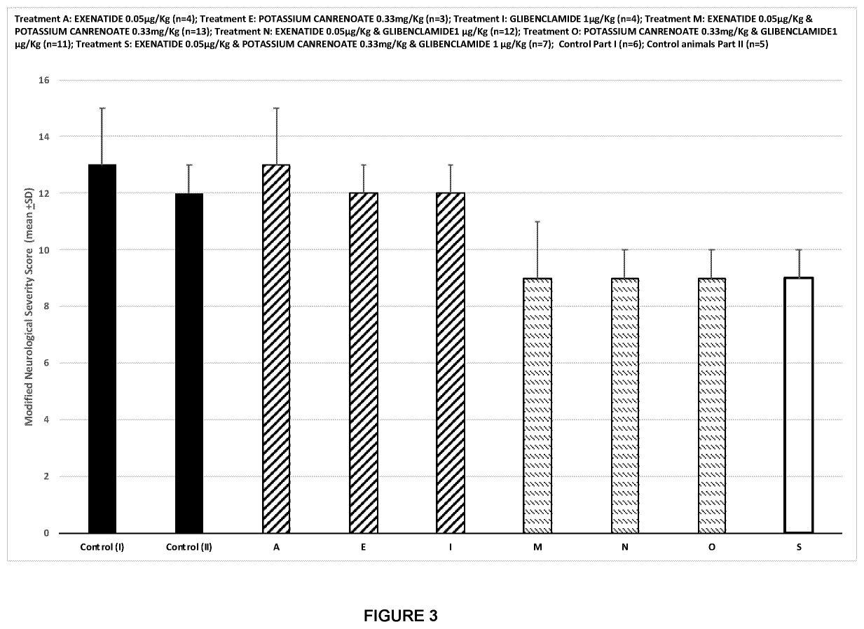

FIG. 3 shows the Modified Neurological Severity Score on Day 7 following repeated administration (7 days) of the low dose triple combination (treatment S) versus that in control animals in Part I and Part II and in animals that received the corresponding monotherapies (treatments A, E, and I) and the corresponding double combinations (treatments M, N, and O) in a rat model of transient middle cerebral artery occlusion. Treatment A: Exenatide 0.05 μg/kg (n=4); Treatment E: Potassium canrenoate 0.33 mg/kg (n=3); Treatment I: Glibenclamide 1 μg/kg (n=4); Treatment M: Exenatide 0.05 μg/kg and Potassium canrenoate 0.33 mg/kg (n=13); Treatment N: Exenatide 0.05 μg/kg and Glibenclamide 1 μg/kg (n=12); Treatment O: Potassium canrenoate 0.33 mg/kg and Glibenclamide 1 μg/kg (n=11); Treatment S: Exenatide 0.05 μg/kg and Potassium canrenoate 0.33 mg/kg and Glibenclamide 1 μg/kg (n=7). Control Part I (n=6); Control animals Part II (n=5).

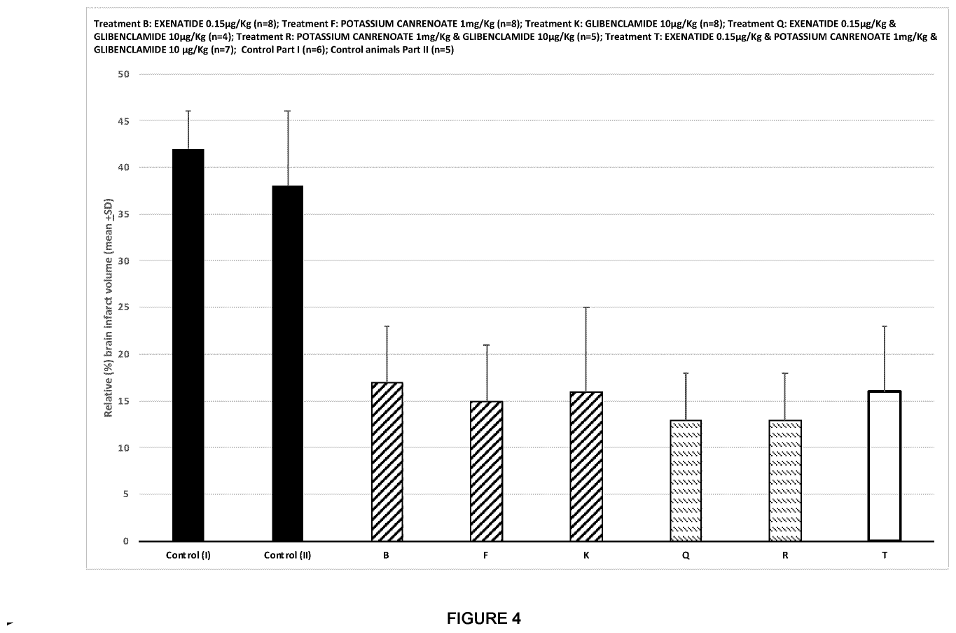

FIG. 4 shows the relative brain infarct volume (%) on Day 7 following repeated administration (7 days) of the higher dose triple combination (treatment T) versus that in control animals in Part I and Part II and in animals that received the corresponding monotherapies (treatments B, F, and K) and the corresponding double combinations (treatments Q, and R) in a rat model of transient middle cerebral artery occlusion. Treatment B: Exenatide 0.15 μg/kg (n=8); Treatment F: Potassium canrenoate 1 mg/kg (n=8); Treatment K: Glibenclamide 10 μg/kg (n=8); Treatment Q: Exenatide 0.15 μg/kg and Glibenclamide 10 μg/kg (n=4); Treatment R: Potassium canrenoate 1 mg/kg and Glibenclamide 10 μg/kg (n=5); Treatment T: Exenatide 0.15 μg/kg and Potassium canrenoate 1 mg/kg and Glibenclamide 10 μg/kg (n=7). Control Part I (n=6); Control animals Part II (n=5).

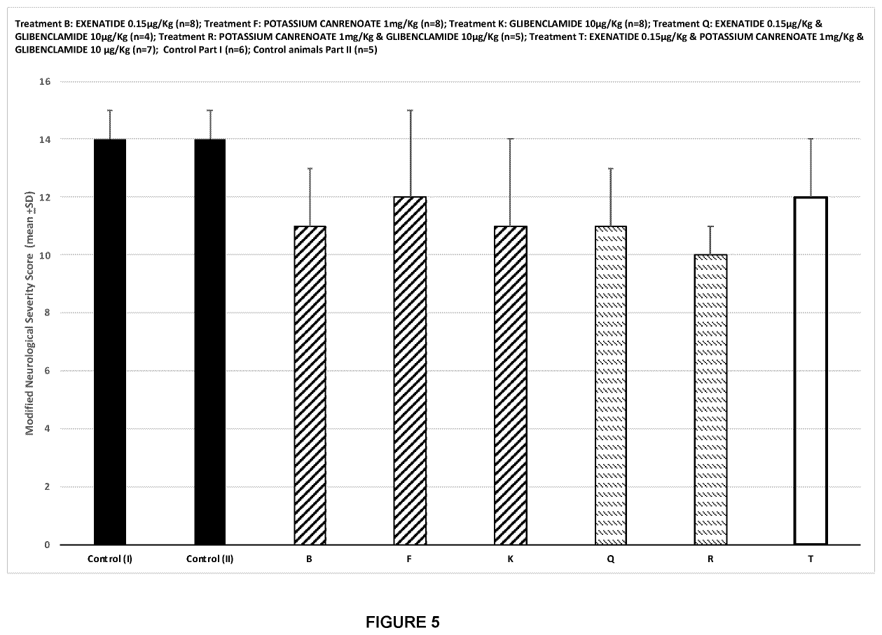

FIG. 5 shows the Modified Neurological Severity Score on Day 2 following repeated administration (7 days) of the higher dose triple combination (treatment T) versus that in control animals in Part I and Part II and in animals that received the corresponding monotherapies (treatments B, F, and K) and the corresponding double combinations (treatments Q, and R) in a rat model of transient middle cerebral artery occlusion. Treatment B: Exenatide 0.15 μg/kg (n=8); Treatment F: Potassium canrenoate 1 mg/kg (n=8); Treatment K: Glibenclamide 10 μg/kg (n=8); Treatment Q: Exenatide 0.15 μg/kg and Glibenclamide 10 μg/kg (n=4); Treatment R: Potassium canrenoate 1 mg/kg and Glibenclamide 10 μg/kg (n=5); Treatment T: Exenatide 0.15 μg/kg and Potassium canrenoate 1 mg/kg and Glibenclamide 10 μg/kg (n=7). Control Part I (n=6); Control animals Part II (n=5).

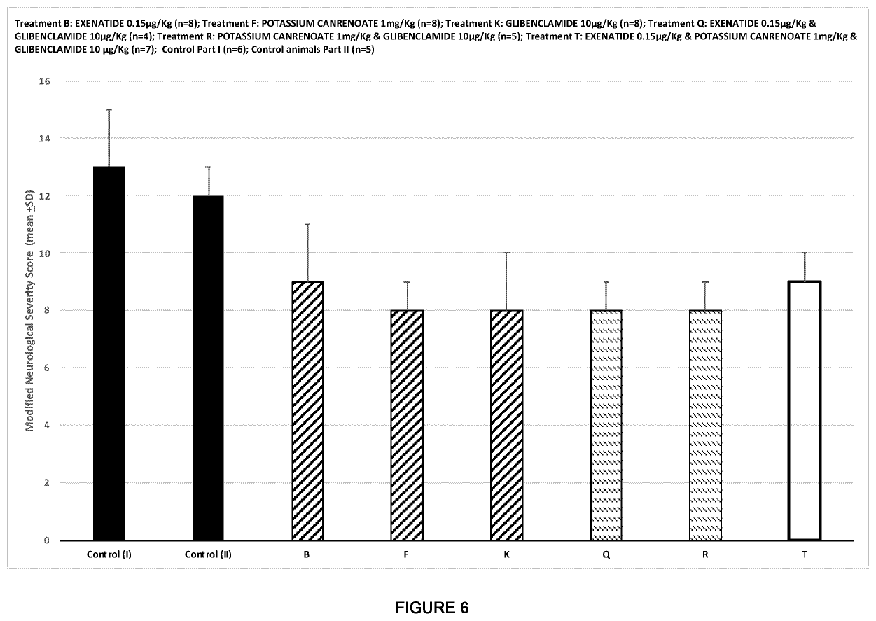

FIG. 6 shows the Modified Neurological Severity Score on Day 7 following repeated administration (7 days) of the higher dose triple combination (treatment T) versus that in control animals in Part I and Part II and in animals that received the corresponding monotherapies (treatments B, F, and K) and the corresponding double combinations (treatments Q, and R) in a rat model of transient middle cerebral artery occlusion. Treatment B: Exenatide 0.15 μg/kg (n=8); Treatment F: Potassium canrenoate 1 mg/kg (n=8); Treatment K: Glibenclamide 10 μg/kg (n=8); Treatment Q: Exenatide 0.15 μg/kg and Glibenclamide 10 μg/kg (n=4); Treatment R: Potassium canrenoate 1 mg/kg and Glibenclamide 10 μg/kg (n=5); Treatment T: Exenatide 0.15 μg/kg and Potassium canrenoate 1 mg/kg and Glibenclamide 10 μg/kg (n=7). Control Part I (n=6); Control animals Part II (n=5).

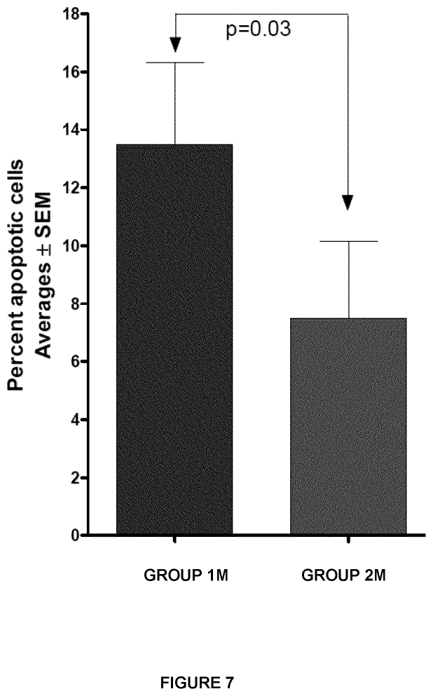

FIG. 7 shows the results of histology TUNEL staining for apoptosis in the hippocampal area of rats treated with the triple combination of exenatide/potassium canrenoate/glibenclamide in a rat model for vascular dementia. More specifically, FIG. 7 shows the percentage of apoptotic cells (average±SEM) for rats treated with exenatide 0.05 μg/kg+potassium canrenoate 0.33 mg/kg+glibenclamide 1 μg/kg (Group 2M; 13 animals; intravenous administration), compared to the vehicle treated control (Group 1M; 9 animals).

### EXAMPLES

The present invention is further illustrated by the following examples, which should not be construed as limiting in any way.

### Example 1. Dose-Response Study of the Efficacy of Exenatide, Potassium Canrenoate and Glibenclamide and their Combinations in a Rat Model of Cerebral Ischemia and Reperfusion Injury

The aim of this study was to assess (a) the dose-response of the neuroprotective effect of glibenclamide, exenatide, and potassium canrenoate in a rat model of cerebral ischemia and reperfusion injury following repeated intravenous administration as monotherapies (Study Part I) and (b) the effect of a combination of the compounds vs the corresponding monotherapies (Study Part II).

The transient middle artery occlusion (t-MCAO) was performed according to the method described by R. Schmid-Elsaesser et al. (Stroke. 1998; 29(10): 2162-70). Test compounds were administrated intravenously 20 minutes before reperfusion and then twice a day thereafter for six consecutive days. The modified neurological severity score (NSS) was graded on a scale of 0 to 18 (in which normal score was 0 and maximal deficit score was represented by 18), on Study Day 2 (one day following surgery) and on Study Day 7 (7 days post-surgery and before study termination); it included a set of clinical-neurological tests (composite of motor, sensory, reflex and balance tests). At study termination brains were harvested, sliced into five 2 mm thick coronal sections and stained with triphenyl tetrazolium chloride (TTC) for infarct size measurement using ImageJ program. Morbidity, mortality, body weight and clinical observation were also recorded. Numerical results are shown as means±standard deviation of the mean. The statistical significance (P) of treated groups vs the untreated control group was determined using two-way ANOVA followed by Bonferroni post hoc test, using GraphPad Prism 5 program.

In study Part I, a total of 112 SD male rats (270-320 gr upon arrival) were divided into 13 groups (5 or 10 rats per treated group and 8 rats in the control group). Due to mortality in some of the groups, the numbers within the groups were adjusted to have sufficient animals in all the groups. Groups were as follows:

CONTROL (saline)
EXENATIDE administered at 0.05 μg/kg, 0.15 μg/kg, 0.5 μg/kg and 1.5 μg/kg;
POTASSIUM CANRENOATE administered at 0.33 mg/kg, 1 mg/kg, 3 mg/kg and 10 mg/kg;
GLIBENCLAMIDE administered at 1 μg/kg, 3 μg/kg, 10 μg/kg and 30 μg/kg.

The results of the efficacy endpoints obtained in Part I are summarised in Table 1, and are also expressed as percentage change vs the corresponding controls including the results of the statistical comparison of each treatment vs the corresponding control groups. Twenty-seven animals died during the study across all groups (1 during the operation, 5 after the occlusion, 1 was euthanised on Day 2, 7 shortly after reperfusion and 13 were found dead in their cages within one-five days after surgery). There was no statistically significant differences in body weight between all animals' groups.

All monotherapies, with the exception of the lowest doses of exenatide, potassium canrenoate and glibenclamide (treatments A, E and I), showed a statistically significant decrease in the brain infarct size when compared with the control group. There was no clear indication of a dose-response.

In respect of the modified neurological severity score (NSS), only the lowest doses of exenatide, potassium canrenoate and glibenclamide (treatments A, E and I) and the 3 μg/kg dose of glibenclamide (treatment J) did not show a statistically significant decrease when compared with the control group on Day 2, while on Day 7, only the lowest doses of exenatide, potassium canrenoate and glibenclamide (treatments A, E and I) and the 0.5 μg/kg dose of exenatide (treatment C) did not show a statistically significant decrease when compared with the control group. As in the case of the brain infarct size, there was no clear indication of a dose-response on either day for the modified NSS.

In study Part II, a total of 86 SD male rats (270-320 gr upon arrival) were divided into 9 groups (7 or 15 rats per treated group and 6 rats in the control group). Due to mortality in some of the groups, the numbers within the groups were adjusted to have sufficient animals in all the groups. Groups were as follows:

CONTROL (saline)
EXENATIDE administered at 0.05 μg/kg, and POTASSIUM CANRENOATE administered at 0.33 mg/kg;
EXENATIDE administered at 0.05 μg/kg, and GLIBENCLAMIDE administered at 1 μg/kg;
POTASSIUM CANRENOATE administered at 0.33 mg/kg and GLIBENCLAMIDE administered at 1 μg/kg;
EXENATIDE administered at 0.15 μg/kg, and POTASSIUM CANRENOATE administered at 0.33 mg/kg;
EXENATIDE administered at 0.15 μg/kg, and GLIBENCLAMIDE administered at 10 μg/kg;
POTASSIUM CANRENOATE administered at 1 mg/kg, and GLIBENCLAMIDE administered at 10 μg/kg;
EXENATIDE administered at 0.05 μg/kg, and POTASSIUM CANRENOATE administered at 0.33 mg/kg and GLIBENCLAMIDE administered at 1 μg/kg;
EXENATIDE administered at 0.15 μg/kg, and POTASSIUM CANRENOATE administered at 1 mg/kg, and GLIBENCLAMIDE administered at 10 μg/kg.

The results of the efficacy endpoints obtained in Part II are summarised in Table 2, and are also expressed as percentage change vs the corresponding controls including the results of the statistical comparison of each treatment vs the corresponding control groups. Nineteen animals died during the study across all groups (2 were euthanised on Day 6, 4 died shortly after reperfusion and 13 were found dead in their cages within one-five days after surgery). There was no statistically significant differences in body weight between all animals' groups.

All double combinations (Groups M, N, O, P, Q and R) as well as both triple combinations (Groups S and T) showed a statistically significant change in the brain infarct size when compared with the control group. There was no difference between the double dose (Groups M, N, O, P, Q and R) or the triple dose combinations (Groups S and T). The decrease in brain infarct size following administration of the double combination of the lowest doses of exenatide and potassium canrenoate (Group M) was statistically different from that of the corresponding monotherapies (Groups A and E). The decrease in brain infarct size following administration of the lowest doses of the double combination of exenatide and glibenclamide (Group N) was statistically different only from that of the corresponding glibenclamide monotherapy (Group I). Furthermore, the triple combination with the lowest doses (Group S), but not that of the higher doses (Group T), was shown to be statistically significant in terms of the decrease in the brain infarct size compared to the corresponding monotherapies.

All double combinations (Groups M, N, O, P, Q, and R) and both triple combinations (Groups S and T) showed a statistically significant decrease in the modified neurological severity score (NSS) when compared with the control group on Day 2 and on Day 7. Furthermore, the decrease in the modified NSS with the triple combination of the lowest doses of exenatide, potassium canrenoate and glibenclamide (Group S) was statistically significantly different to the corresponding potassium canrenoate and glibenclamide monotherapies (Groups E and I) on Day 2 and to all three corresponding monotherapies (Groups A, E and I) on Day 7, but not to any of the corresponding double combinations (Groups M, N and O). The triple combination of the higher doses of exenatide, potassium canrenoate and glibenclamide (Group T) was not statistically significantly different to any of the corresponding double combinations (Groups Q, and R).

To appreciate the effect of the combination therapies, the triple combination of the lowest doses (treatment “S”) is shown in comparison with the corresponding monotherapies and double combinations in FIGS. 1-3, for the relative brain infarct volume, and the modified neurological severity score on Days 2 and 7, respectively. Similarly, the results of the comparison of the triple combination of the higher doses (treatment “T”) vs the corresponding monotherapies and double combinations are shown in FIGS. 4-6.

From the results obtained it is clear that low doses of exenatide, potassium canrenoate and glibenclamide, which are ineffective when administered as monotherapies, show a statistically significant efficacy when they are combined (both as double or triple combinations), indicative of a synergistic effect. The present results provide strong evidence that the combination therapy of glibenclamide with exenatide and/or potassium canrenoate
reduced the extent of cerebral infarction and/or
improved the neurological severity score and/or
improved the motor performance score in a synergistic manner,
   since the obtained combination effect exceeded the sum of the respective monotherapies' effects. Furthermore, the surprising finding was that the dose of glibenclamide, which produced this synergistic effect in the present study (i.e. 1 μg/kg twice daily that is 0.66 μg in the rats weighing 330 g used in the study), was significantly lower than the dose previously reported in the literature for stroke, i.e. daily infusions of 200 ng/h, that is 4.8 μg (Simard et al, Transl Stroke Res. 2012). Importantly, such very low doses of glibenclamide when used in the context of the present invention correspond to a dose (i.e. 70 μg/day) that is 100 times less than the defined daily dose (7 mg—orally of the micronized formulation) or ˜20 to 285-fold less than the recommended maintenance dose of glibenclamide (micronized formulation), and are therefore expected to be devoid of any effects on blood glucose levels or of any adverse effects. The above clinically effective dose of glibenclamide as a double or triple combination with low doses of exenatide and/or potassium carbonate is also significantly lower than the dose of glibenclamide shown to be neuroprotective (continuous infusions of 0.16 or 0.11 mg/h, that is 3.84 mg or 2.64 mg daily) in the clinical studies published in the literature (see King Z A et al).

### Example 2. Study of the Efficacy of Exenatide, Potassium Canrenoate and Glibenclamide Combination in a Rat Model of Vascular Dementia

Chronic cerebral hypoperfusion model in Wistar rat causes cerebral lesions in the rat brain by permanent occlusion of both common carotid arteries which can also affect cognitive functional deficit. This model is similar to that of Vascular Dementia and the technique can decrease the blood flow in the cerebral cortex and hippocampus by up to 40-80% for several months, which induces certain learning disorders.

### Study Objective

The purpose of the study was to evaluate the neuroprotective efficacy of a combination of exenatide, potassium canrenoate and glibenclamide, given intravenously 24 h after both common carotid arteries permanent ligation and then administered twice daily for three weeks, using the Wistar rat Vascular Dementia model.

### Treatment Groups

Treatment Groups were follows:

Group 1M: Vehicle treated controls (9 animals, intravenous administration);
Group 2M: Exenatide 0.05 μg/kg+potassium canrenoate 0.33 mg/kg+glibenclamide 1 μg/kg (13 animals; intravenous administration).

Exenatide acetate salt was obtained from Bachem AG, Switzerland. Potassium canrenoate was obtained from Pfizer, Switzerland. Glibenclamide was obtained from Tocris Bioscience.

### Study Design and Timeline

This study evaluated the neuroprotective effect of the combination administered at a low dose intravenously twice a day during three weeks in the Wistar rat Vascular Dementia model. Test compounds were administrated 24 hours after common carotid arteries ligation twice a day for three weeks. On Day 1 both common carotid arteries were permanently ligated. Morris water maze tests were performed before common carotid arteries ligation as training for baseline and on Week 4 and Week 8 thereafter. At study termination brains were harvested. Histological analysis was performed for the tissues.

The study timeline was as follows:

| Study Day/Week | | CCAO | BW | Clinical Observation | MWM | Treatment | Termination |
|---|---|---|---|---|---|---|---|
| On the week | | | ✓ | ✓ | ✓ | | |
| before surgery | | | | | (training) | | |
| D 1 | Surgery | ✓ | ✓ | 4 hr post-surgery, | | 24 hours after common | |

| | | | | | |
|---|---|---|---|---|---|
| Week 4 (W 4) | Twice | twice a day during | ✓ | carotid arteries ligation, | |
| | a week | first two days, | | twice a day for three weeks | |
| Week 8 (W 8) | | then twice a week | ✓ | | ✓ |

CCAO = Common Carotid Arteries Occlusion;
D = Day;
W = Week;
BW = Body Weight;
MWM = Morris Water Maze

First dosing day was assigned “Day 1” and termination was “Day 56”, eight weeks following common carotid arteries ligation.

### Histology Analysis

Tissue preparation and trimming (affected hemisphere), X3 accurate cross sections of the striatum (Corpus Callosum) dorsal hippocampus and optical trac per brain. Paraffin block preparation H&E and TUNEL staining, IHC: Double Cortin for neuro-regeneration in brain sub-ventricular zone. MBP, myelin in white matter, lba-1 for microglia and GFAP for astrocytes. Olig-2 for all Oligodendrocytes, NG2 for young Oligodendrocytes. Slides evaluation analysis; cell bodies counting at hippocampal CA1 and CA3 regions—three sections per brain, three fields per section Morphometric analysis of neuronal death count and MBP.

### Animals

Male Wistar rats were used in the study, weighing 290-390 g at study initiation.

### Animal Management

### Housing

Animal handling was performed according to guideline of the National Institute of Health (NIH) and the Association for Assessment and Accreditation of Laboratory Animal Care (AAALAC). Animals were housed in polyethylene cages (maximum 3 rats/cage) measuring 42.5×26.5×18.5 cm, with stainless steel top grill facilitating pelleted food and drinking water in plastic bottle; bedding: steam sterilized clean paddy husk (Envigo, Sani-chips cat #7090C). Bedding material was changed along with the cage at least twice a week.

### Diet

Animals were fed ad libitum a commercial rodent diet (Teklad Certified Global 18% Protein Diet, Envigo cat #2018SC). Animals had free access to standard tap drinking water obtained from the municipality supply and treated according to Pharmaseed's SOP No. 214: “Water system”. Animal feed arrived with a certificate of analysis and the water was autoclaved prior to use.

### Environment Conditions

Animals were singly housed for the period following surgery in a climate-controlled environment. Air was filtered (HEPA F6/6) with adequate fresh supply (minimum of 15 air changes/hour). Temperatures were maintained at 18-24° C. and relative humidity 30-70%. Animals were exposed to 12-hour light and 12-hour dark cycles (6 AM/6 PM).

### Randomization

Animals were randomly allocated into cages according to Pharmaseed's SOP #027 “Random allocation of animals”.

### Procedures and Evaluations

### Surgical Procedure: Two Common Carotid Arteries Ligation

On the day of surgery anesthesia was induced on a heating pad with 4% isoflurane in a mixture of 70% N₂O and 30% O₂ and maintained with 1.5-2% isoflurane. Buprenorphine at 0.1 mg/kg was injected subcutaneously. Two common carotid artery occlusions were performed according to the method described by Hyun Joon Lee et al (Citicoline Protects Against Cognitive Impairment in a Rat Model of Chronic Cerebral Hypoperfusion, J Clin Neurol. 2009; 5(1):33-38). Both Common Carotid Arteries (CCA) were exposed through a midline neck incision and carefully dissected free from surrounding nerves and fascia. Both arteries were double ligated with a 4-0 silk suture at 8-10 mm below the visible region of the external carotid artery. The surgical wound was closed and the animals returned to their cages to recover from anesthesia. Analgesic treatment with Buprenorphine was given again by the end of the day and twice a day during the next four days.

### Administration of Combination

Treatment started 24 hours after arteries ligation, via intravenous (IV) injection. Treatment was performed twice a day for three consecutive weeks.

### Body Weight

Animals' body weight was monitored during acclimation, before common carotid artery ligation and twice a week thereafter. Animals were weighed according to Pharmaseed's SOP No. 010: “Weighing laboratory animals”. Individual body weight changes were calculated.

### Clinical Observation

Clinical signs were monitored once during acclimation, for the first 4 h post-surgery, twice a day during the first two days following surgery, then twice a week.

### Morris Water Maze Test

The Morris water maze (MWM) test is designed to assess cognitive deficits following common carotid arteries ligation. The test was performed according to Pharmaseed's SOP 100 (Morris Water Maze Testing V6) and related publications (e.g. Brandeis R, Brandys Y and Yehuda S, “The use of the Morris Water Maze in the study of memory and learning”, Int J Neurosci. 1989; 48(1-2):29-69).

### Pre Surgery Training

Animals were trained and conditioned for one week in the Morris Water Maze, according to Pharmaseed's SOP 100 and the scientific publications (e.g. see Brandeis R et al). Before MWM, rats' cages were transferred from the animal housing to the behaviour testing room for an acclimation of about one hour.

The training results on the last day were considered as baseline data for comparison. MWM test had exclusion criteria as follows: failing to escape to the platform in 90 sec. (on Day 3 of training).

### Post Common Carotid Arteries Ligation Testing

Before MWM, rats' cages were transferred from the animal housing to the behaviour testing room for an acclimation of about one hour.

The MWM test was performed on Week 4 and 8 after common carotid arteries ligation.

### Statistical Analysis

Numerical results are given as means and standard deviations or standard errors. Descriptive statistics and group comparisons of data were performed, whenever possible, using statistical analysis program (GraphPad Prism version 5.02 for Windows, GraphPad Software, San Diego Calif. USA). The appropriate parametric or non-parametric test was performed followed by the appropriate post-hoc analysis. A probability of 5% (p≤0.05) is regarded as statistically significant.

### Results

Preliminary results for the MWM test indicate that animals treated with the triple combination of exenatide/potassium/glibenclamide (treatment Group 2M; 13 animals) performed better than vehicle treated animals (Group 1M; 9 animals) both in terms of the average time taken to reach the platform (sec), and the average distance swim to the platform (cm). Furthermore, quantitative evaluation of histological TUNEL staining for apoptosis in the hippocampal area exhibited statistically significant neuroprotection (at p=0.03 according to the Mann Whitney test) for treatment Group 2M over Group 1M (vehicle). FIG. 7 shows the percentage apoptotic cells (average±SEM) for each group.

Thus, it can be concluded that the triple combination of exenatide/potassium/glibenclamide when administered at a low dose intravenously twice a day for three weeks exhibited a neuroprotective effect in the Wistar rat Vascular Dementia model, showing both improved cognitive behavior and reduced apoptosis in the hippocampal brain area.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following claims.

### REFERENCES

Abdallah D M, et al. Glibenclamide ameliorates ischemia-reperfusion injury via modulating oxidative stress and inflammatory mediators in the rat hippocampus. Brain Res. 2011; 1385: 257-62.
Atri A. The Alzheimer's Disease Clinical Spectrum: Diagnosis and Management. Med Clin North Am. 2019; 103: 263-293.
Bai J, Lyden P D. Revisiting cerebral post-ischemic reperfusion injury: new insights in understanding reperfusion failure, hemorrhage, and edema. Int J Stroke. 2015; 10: 143-52.
Battistin L, Cagnin A (December 2010). “Vascular cognitive disorder. A biological and clinical overview”. Neurochemical Research. 35 (12): 1933-8. doi:10.1007/s11064-010-0346-5. PMID 21127967.
Brandeis R, Brandys Y and Yehuda S, “The use of the Morris Water Maze in the study of memory and learning”, Int J Neurosci. 1989; 48(1-2):29-69.
Berge S M, et al. Pharmaceutical salts. J Pharm Sci. 1977; 66: 1-19.
Beygui F, et al. High plasma aldosterone levels on admission are associated with death in patients presenting with acute ST-elevation myocardial infarction. Circulation. 2006; 114: 2604-10.
Bovelli D, et al. Cardiotoxicity of chemotherapeutic agents and radiotherapy-related heart disease: ESMO Clinical Practice Guidelines. Ann Oncol. 2010; 21 (Supp) 5): v277-82.
Bullock B P, et al. Tissue distribution of messenger ribonucleic acid encoding the rat glucagon-like peptide-1 receptor. Endocrinology. 1996; 137: 2968-78.
Caffes N, et al. Glibenclamide for the treatment of ischemic and hemorrhagic stroke. Int J Mol Sci. 2015; 16: 4973-84.
Chaudhuri K R, et al. Unmet needs in Parkinson's disease: New horizons in a changing landscape. Parkinsonism Relat Disord. 2016; 33 (Supp) 1): S2-S8.
Cordiner R L M, Pearson E R. Reflections on the sulfonylurea story: A drug class at risk of extinction or a drug class worth reviving? Diabetes Obes Metab. 2019; 21: 761-771
Dae M W, et al. Effect of endovascular cooling on myocardial temperature, infarct size, and cardiac output in human-sized pigs. Am J Physiol Heart Circ Physiol. 2002; 282: H1584-91.
Dinh Q N, et al. Cell-specific mineralocorticoid receptors: future therapeutic targets for stroke? Neural Regen Res. 2016; 11(8):1230-1.
Garber A J, et al. CONSENSUS STATEMENT BY THE AMERICAN ASSOCIATION OF CLINICAL ENDOCRINOLOGISTS AND AMERICAN COLLEGE OF ENDOCRINOLOGY ON THE COMPREHENSIVE TYPE 2 DIABETES MANAGEMENT ALGORITHM—2019 EXECUTIVE SUMMARY. Endocr Pract. 2019; 25: 69-100.
Göke R, et al. Exendin-4 is a high potency agonist and truncated exendin-(9-39)-amide an antagonist at the glucagon-like peptide 1-(7-36)-amide receptor of insulin-secreting beta-cells. J. Biol. Chem. 268: 19650-19655.
Götberg M, et al. Mild hypothermia reduces acute mortality and improves hemodynamic outcome in a cardiogenic shock pig model. Resuscitation. 2010; 81: 1190-6.
Gouzoulis-Mayfrank E, Daumann J. Neurotoxicity of drugs of abuse—the case of methylenedioxyamphetamines (MDMA, ecstasy), and amphetamines. Dialogues Clin Neurosci. 2009; 11: 305-17.
Gribble F M, Reimann F. Sulfonylurea action revisited: the post-cloning era. Diabetologia. 2003; 46: 875-891.
Hambrock A, et al. Resveratrol binds to the sulfonylurea receptor (SUR) and induces apoptosis in a SUR subtype-specific manner. J Biol Chem. 2007; 282: 3347-56.
Hemphill J C 3rd, et al. Guidelines for the management of spontaneous intracerebral hemorrhage: a guideline for healthcare professionals from the American Heart Association/American Stroke Association. Stroke. 2015; 46: 2032-2060.
Hochman J S, et al. Early revascularization in acute myocardial infarction complicated by cardiogenic shock. SHOCK Investigators. Should We Emergently Revascularize Occluded Coronaries for Cardiogenic Shock. N Engl J Med. 1999; 341: 625-34.
Holmes D R Jr, et al. Contemporary reperfusion therapy for cardiogenic shock: the GUSTO-I trial experience. The GUSTO-I Investigators. Global Utilization of Streptokinase and Tissue Plasminogen Activator for Occluded Coronary Arteries. J Am Coll Cardiol. 1995; 26: 668-74.
Huang K, et al. Exploratory Analysis of Oral Glibenclamide in Acute Ischemic Stroke. Acta Neurol Scand. 2019 May 29; doi: 10.1111/ane.13134.
Inzucchi S E, et al. Management of hyperglycemia in type 2 diabetes, 2015: a patient-centered approach: update to a position statement of the American Diabetes Association and the European Association for the Study of Diabetes. Diabetes Care. 2015; 38: 140-9.
Jiang B, et al. Role of Glibenclamide in Brain Injury After Intracerebral Hemorrhage. Transl Stroke Res. 2017; 8: 183-193.
Johnson C O et al. Global, regional, and national burden of stroke, 1990-2016: a systematic analysis for the Global Burden of Disease Study 2016. Lancet Neurol. 2019; 18: 439-458.
Jorm A F, Korten A E, Henderson A S (November 1987). “The prevalence of dementia: a quantitative integration of the literature”. Acta Psychiatrica Scandinavica. 76 (5): 465-79. doi:10.1111/j.1600-0447.1987.tb02906.x. PMID 3324647.
Kern K B. Usefulness of cardiac arrest centers—extending lifesaving post-resuscitation therapies: the Arizona experience. Circ J. 2015; 79(6): 1156-63.
Kim J Y, et al. Multilayer nanoparticles for sustained delivery of exenatide to treat type 2 diabetes mellitus. Biomaterials. 2013; 34: 8444-9.
King Z A, et al. Profile of intravenous glyburide for the prevention of cerebral edema following large hemispheric infarction: evidence to date. Drug Des Devel Ther. 2018; 12: 2539-2552.
Kronick S L, et al. Part 4: Systems of Care and Continuous Quality Improvement: 2015 American Heart Association Guidelines Update for Cardiopulmonary Resuscitation and Emergency Cardiovascular Care. Circulation. 2015; 132 (18 Suppl 2): S397-413.
Kunte H, et al. Hemorrhagic transformation of ischemic stroke in diabetics on sulfonylureas. Ann Neurol. 2012; 72: 799-806.
Hyun Joon Lee et al (Citicoline Protects Against Cognitive Impairment in a Rat Model of Chronic Cerebral Hypoperfusion, J Clin Neurol. 2009; 5(1):33-38.
Lopez M S, et al. Resveratrol neuroprotection in stroke and traumatic CNS injury. Neurochem Int. 2015; 89: 75-82.
Marin B, et al. Variation in worldwide incidence of amyotrophic lateral sclerosis: a meta-analysis. Int J Epidemiol 2017; 46: 57-74.
Mehta R I et al. Sur1-Trpm4 Cation Channel Expression in Human Cerebral Infarcts. J Neuropathol Exp Neurol. 2015; 74: 835-49.
Nishida H et al. Glimepiride treatment upon reperfusion limits infarct size via the phosphatidylinositol 3-kinase/Akt pathway in rabbit hearts. J Pharmacol Sci. 2009; 109: 251-6.
Nystrom T, et al. Effects of glucagon-like peptide-1 on endothelial function in type 2 diabetes patients with stable coronary artery disease. Am J Physiol Endocrinol Metab. 2004; 287: E1209-15.
Okuda M. A multidisciplinary overview of cardiogenic shock. Shock. 2006; 25: 557-70.
Patel A D, et al. Glibenclamide reduces hippocampal injury and preserves rapid spatial learning in a model of traumatic brain injury. J Neuropathol Exp Neurol. 2010; 69: 1177-90.
Patel R A G, et al. Neuroprotection in the Treatment of Acute Ischemic Stroke. Prog Cardiovasc Dis. 2017; 59: 542-548.
Patel R D, Saver J L. Evolution of reperfusion therapies for acute brain and acute myocardial ischemia: a systematic, comparative analysis. Stroke. 2013; 44: 94-8.
Powers W J, et al. 2018 Guidelines for the early management of patients with acute ischemic stroke: a guideline for healthcare professionals from the American Heart Association/American Stroke Association. Stroke. 2018; 49: e46-e99.
Plassman B L, Langa K M, Fisher G G, Heeringa S G, Weir D R, Ofstedal M B, Burke J R, Hurd M D, Potter G G, Rodgers W L, Steffens D C, Willis R J, Wallace R B (2007). “Prevalence of dementia in the United States: the aging, demographics, and memory study”. Neuroepidemiology. 29 (1-2): 125-32. doi:10.1159/000109998. PMC 2705925. PMID 17975326.
Proks P, et al. Sulfonylurea stimulation of insulin secretion. Diabetes. 2002; 51 (Supp) 3): S368-76.
Rawlins M D. The Prevalence of Huntington's Disease. Neuroepidemiology. 2016; 46: 144-53.
Roth G A, et al. Global, regional, and national age-sex-specific mortality for 282 causes of death in 195 countries and territories, 1980-2017: a systematic analysis for the Global Burden of Disease Study 2017. Lancet. 2018; 392:1736-1788.
Savitz S I, et al. Reconsidering Neuroprotection in the Reperfusion Era. Stroke. 2017; 48: 3413-3419.
Schmid-Elsaesser R, et al. A critical reevaluation of the intraluminal thread model of focal cerebral ischemia: evidence of inadvertent premature reperfusion and subarachnoid hemorrhage in rats by laser-Doppler flowmetry. Stroke. 1998; 29: 2162-70.
Simard J M, et al. Newly expressed SUR1-regulated NC(Ca-ATP) channel mediates cerebral edema after ischemic stroke. Nat Med. 2006; 12: 433-40.
Simard, J M, et al. Glibenclamide reduces inflammation, vasogenic edema, and caspase-3 activation after subarachnoid hemorrhage. Journal of Cerebral Blood Flow and Metabolism. 2009; 29; 317-330.
Simard J M, et al. Glibenclamide-10-h Treatment Window in a Clinically Relevant Model of Stroke. Transl Stroke Res. 2012; 3: 286-95.
Simard, J M, et al. Does inhibiting Sur1 complement rt-PA in cerebral ischemia? Ann. N. Y. Acad. Sci. 2012, 1268, 95-107.
Sola D, et al. Sulfonylureas and their use in clinical practice. Arch Med Sci. 2015; 11: 840-8.
Struthers A D. Aldosterone: cardiovascular assault. Am Heart J. 2002; 144 (5 Suppl): S2-7.
Tan F, et al. Protective effect of treatment with low-dose gliclazide in a model of middle cerebral artery occlusion and reperfusion in rats. Brain Res. 2014; 1560: 83-90.
Tosun C, et al. Inhibition of the Sur1-Trpm4 channel reduces neuroinflammation and cognitive impairment in subarachnoid hemorrhage. Stroke. 2013; 44: 3522-8.
Wilkinson C M, et al. Glibenclamide, a Sur1-Trpm4 antagonist, does not improve outcome after collagenase-induced intracerebral hemorrhage. PLoS One. 2019; 14: e0215952.
Woo S K, et al. The sulfonylurea receptor 1 (Sur1)-transient receptor potential melastatin 4 (Trpm4) channel. J Biol Chem. 2013; 288: 3655-67.
Xu Z M, et al. Glibenclamide Attenuates Blood-Brain Barrier Disruption in Adult Mice after Traumatic Brain Injury. J Neurotrauma. 2017; 34: 925-933.
Xu F, et al. Glibenclamide ameliorates the disrupted blood-brain barrier in experimental intracerebral hemorrhage by inhibiting the activation of NLRP3 inflammasome. Brain Behay. 2019; 9: e01254.
Zannad F, Radauceanu A. Effect of MR blockade on collagen formation and cardiovascular disease with a specific emphasis on heart failure. Heart Fail Rev. 2005; 10: 71-8.
Zhang G, et al. A Protective Role of Glibenclamide in Inflammation-Associated Injury. Mediators Inflamm. 2017; 2017: 3578702.
Zhu S, et al. Glibenclamide Enhances the Therapeutic Benefits of Early Hypothermia after Severe Stroke in Rats. Aging Dis. 2018; 9: 685-695.

TABLE 1
Effects of exenatide, potassium canrenoate and glibenclamide on cerebral
infarction on Day 7 and on the modified neurological severity score (NSS)
on Days 2 and 7, following repeated administration (7 days) as monotherapies
in a rat model of transient middle cerebral artery occlusion.

| Treatment & Dose | | Relative brain infarct | Modified NSS | Modified NSS |
|---|---|---|---|---|
| (number of animals at | | volume on Day 7 (%) | on Day 2 | on Day 7 |
| start/on Day 2/on Day 7) | | (mean + SD) | (mean + SD) | (mean + SD) |
| CONTROL (n = 8/6/6) | | 42 ± 4 | 14 ± 1 | 13 ± 2 |
| A. | EXENATIDE 0.05 μg/Kg | 28 ± 5 | 13 ± 2 | 13 ± 2 |
| | (n = 5/4/4) | [−33%; NS] | [−7%; NS] | [0%; NS] |
| B. | EXENATIDE 0.15 μg/Kg | 17 ± 6 | 11 ± 2 | 9 ± 2 |
| | (n = 10/9/8) | [−60%; ***] | [−21%; **] | [−31%; ***] |
| C. | EXENATIDE 0.5 μg/Kg | 19 ± 12 | 11 ± 2 | 11 ± 3 |
| | (n = 10/7/6) | [−55%; ***] | [−21%; *] | [−15%; NS] |
| D. | EXENATIDE 1.5 μg/Kg | 16 ± 7 | 10 ± 1 | 10 ± 2 |
| | (n = 10/10/10) | [−62%; ***] | [−29%; ***] | [−23%; **] |
| E. | POTASSIUM CANRENOATE | 36 ± 6 | 15 ± 1 | 12 ± 1 |
| | 0.33 mg/Kg (n = 5/4/3) | [−14%; NS] | [7%; NS] | [−8%; NS] |
| F. | POTASSIUM CANRENOATE | 15 ± 6 | 12 ± 3 | 8 ± 1 |
| | 1 mg/Kg (n = 10/8/8) | [−64%; ***] | [−14%; *] | [−38%; ***] |
| G. | POTASSIUM CANRENOATE | 28 ± 11 | 11 ± 2 | 10 ± 2 |
| | 3 mg/Kg (n = 9/8/7) | [−33%; **] | [−21%; *] | [−23%; *] |
| H. | POTASSIUM CANRENOATE | 12 ± 3 | 10 ± 1 | 8 ± 1 |
| | 10 mg/Kg (n = 10/8/7) | [−71%; ***] | [−29%; **] | [−38%; ***] |
| I. | GLIBENCLAMIDE 1 μg/Kg | 33 ± 10 | 14 ± 1 | 12 ± 1 |
| | (n = 5/4/4) | [−21%; NS] | [0%; NS] | [−8%; NS] |
| J. | GLIBENCLAMIDE 3 μg/Kg | 19 ± 9 | 13 ± 3 | 10 ± 1 |
| | (n = 10/7/7) | [−55%; ***] | [−7%; NS] | [−23%; *] |
| K. | GLIBENCLAMIDE 10 μg/Kg | 16 ± 9 | 11 ± 3 | 8 ± 2 |
| | (n = 10/8/8) | [−62%; ***] | [−21%; **] | [−38%; ***] |
| L. | GLIBENCLAMIDE 30 μg/Kg | 17 ± 8 | 11 ± 3 | 8 ± 1 |
| | (n = 10/9/7) | [−60%; ***] | [−21%; **] | [−38%; ***] |

In brackets is shown the percentage change of the effect in the treatment groups vs the effect in the control group, and the result of the statistical comparison of the effect in the treatment groups vs the control group
(NS: not statistically significant;
* p < 0.05;
** p < 0.01;
*** p < 0.001)

TABLE 2
Effects of exenatide, potassium canrenoate and glibenclamide on cerebral infarction
on Day 7 and on the modified neurological severity score (NSS) on Days 2
and 7, following repeated administration (7 days) as double or triple combinations
in a rat model of transient middle cerebral artery occlusion.

| Treatment & Dose | | Relative brain infarct | Modified NSS | Modified NSS |
|---|---|---|---|---|
| (number of animals at | | volume on Day 7 (%) | on Day 2 | on Day 7 |
| start/on Day 2/on Day 7) | | (mean + SD) | (mean + SD) | (mean + SD) |
| CONTROL (n = 6/5/5) | | 38 ± 8 | 14 ± 1 | 12 ± 1 |
| M. | EXENATIDE 0.05 μg/Kg & | 13 ± 2 | 11 ± 1 | 9 ± 2 |
| | POTASSIUM CANRENOATE | [−66%; ***] | [−21%; ***] | [−25%; ***] |
| | 0.33 mg/Kg (n = 15/15/13) | | | |
| N. | EXENATIDE 0.05 μg/Kg & | 17 ± 4 | 11 ± 1 | 9 ± 1 |
| | GLIBENCLAMIDE 1 μg/Kg | [−55%; ***] | [−21%; ***] | [−25%; ***] |
| | (n = 15/12/12) | | | |
| O. | POTASSIUM CANRENOATE | 21 ± 8 | 11 ± 2 | 9 ± 1 |
| | 0.33 mg/Kg & | [−45%; **] | [−21%; ***] | [−25%; ***] |
| | GLIBENCLAMIDE 1 μg/Kg | | | |
| | (n = 15/14/11) | | | |
| P. | EXENATIDE 0.15 μg/Kg & | 11 ± 1 | 11 ± 1 | 9 ± 1 |
| | POTASSIUM CANRENOATE | [−71%; ***] | [−21%; **] | [−25%; **] |
| | 0.33 mg/Kg (n = 7/4/4) | | | |
| Q. | EXENATIDE 0.15 μg/Kg & | 13 ± 5 | 11 ± 2 | 8 ± 1 |
| | GLIBENCLAMIDE 10 μg/Kg | [−66%; ***] | [−21%; **] | [−33%; ***] |
| | (n = 7/5/4) | | | |
| R. | POTASSIUM CANRENOATE | 13 ± 5 | 10 ± 1 | 8 ± 1 |
| | 1 mg/Kg & | [−66%; ***] | [−29%; ***] | [−33%; ***] |
| | GLIBENCLAMIDE 10 μg/Kg | | | |
| | (n = 7/5/5) | | | |
| S. | EXENATIDE 0.05 μg/Kg & | 12 ± 3 | 11 ± 1 | 9 ± 1 |
| | POTASSIUM CANRENOATE | [−68%; ***] | [−21%; **] | [−25%; **] |
| | 0.33 mg/Kg & | | | |
| | GLIBENCLAMIDE 1 μg/Kg | | | |
| | (n = 7/7/7) | | | |
| T. | EXENATIDE 0.15 μg/Kg & | 16 ± 7 | 12 ± 2 | 9 ± 1 |
| | POTASSIUM CANRENOATE | [−58%; ***] | [−14%; *] | [−25%; ***] |
| | 1 mg/Kg & | | | |
| | GLIBENCLAMIDE 10 μg/Kg | | | |
| | (n = 7/7/6) | | | |

In brackets is shown the percentage change of the effect in the treatment groups vs the effect in the control group, and the result of the statistical comparison of the effect in the treatment groups vs the control group
(NS: not statistically significant;
* p < 0.05;
** p < 0.01;
*** p < 0.001)

1. A combination comprising:
(a) a sulfonylurea; and
(b) at least one of the following components: (i) an insulin modulator, and (ii) an aldosterone antagonist.

2. A combination according to claim 1 which comprises a sulfonylurea and an aldosterone antagonist.

3. A combination according to claim 1 which comprises a sulfonylurea and an insulin modulator.

4. A combination according to claim 1 which comprises a sulfonylurea, an insulin modulator, and an aldosterone antagonist.

5. A combination according to any preceding claim wherein the insulin modulator is selected from exenatide, and structural and functional analogues thereof, and pharmaceutically acceptable salts thereof.

6. A combination according to any preceding claim wherein the sulfonylurea is selected from glibenclamide, and structural and functional analogues thereof.

7. A combination according to claim 5 wherein the exenatide structural or functional analogue is a GLP-1 receptor agonist.

8. A combination according to claim 5 wherein the exenatide structural or functional analogue is selected from lixisenatide, albiglutide, liraglutide, taspoglutide and dulaglutide (LY2189265).

9. A combination according to claim 6 wherein the glibenclamide structural or functional analogue is selected from acylhydrazone, sulfonamide and sulfonylthiourea derivatives of glibenclamide, glimepiride, glipizide and gliclazide, preferably gliclazide.

10. A combination according to any preceding claim wherein the aldosterone antagonist is selected from spironolactone, eplerenone, canrenone, potassium canrenoate, finerenone and prorenone, and pharmaceutically acceptable salts thereof where applicable.

11. A combination according to claim 10 wherein the aldosterone antagonist is potassium canrenoate or a structural or functional analogue thereof.

12. A combination according to any preceding claim which comprises at least one further active pharmaceutical ingredient (API) selected from a beta blocker, a renin-angiotensin inhibitor, a statin (HMG-CoA reductase inhibitor), an inhibitor of platelet activation or aggregation, a phosphodiesterase-3 inhibitor, a calcium sensitizer, an antioxidant and an anti-inflammatory agent.

13. A pharmaceutical composition comprising a combination according to any preceding claim and a pharmaceutically acceptable carrier, diluent or excipient.

14. A pharmaceutical composition according to claim 13 in a form suitable for parenteral administration, preferably intravenous administration.

15. A pharmaceutical product comprising:
(a) a sulfonylurea; and
(b) at least one of the following components: (i) an insulin modulator, and (ii) an aldosterone antagonist.

16. A pharmaceutical product according to claim 15 comprising:
(a) glibenclamide, or a structural or functional analogue thereof; and
(b) at least one of the following components: (i) exenatide, or a structural or functional analogue thereof, or a pharmaceutically acceptable salt thereof; and (ii) potassium canrenoate, or a structural or functional analogue thereof.

17. A pharmaceutical product according to claim 16 which comprises glibenclamide and exenatide, or a pharmaceutically acceptable salt thereof.

18. A pharmaceutical product according to claim 16 which comprises glibenclamide and potassium canrenoate.

19. A pharmaceutical product according to claim 16 which comprises glibenclamide, potassium canrenoate and exenatide, or a pharmaceutically acceptable salt thereof.

20. A combination according to any one of claims 1 to 12 or a pharmaceutical composition according to any one of claims 13 and 14 for use in the treatment and/or prevention of one or more of ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection.

21. A combination or a pharmaceutical composition for use according to claim 20 wherein the ischemia and/or reperfusion injury is ischemia and/or reperfusion injury of the brain, heart, lung, kidney, preferably cerebral ischemia, cerebral reperfusion injury or stroke.

22. A combination or a pharmaceutical composition for use according to any one of claims 20 to 21 wherein the components are for administration intravenously.

23. A combination or a pharmaceutical composition for use according to any one of claims 20 to 22 wherein the components are for administration during reperfusion.

24. A combination or a pharmaceutical composition for use according to any one of claims 20 to 22 wherein the components are for administration before reperfusion.

25. A combination or a pharmaceutical composition for use according to any one of claims 20 to 22 wherein the components are for administration after reperfusion.

26. A pharmaceutical product according to any one of claims 15 to 19 for use in the treatment and/or prevention of one or more of the following: ischemia and/or reperfusion injury, stroke, neurodegenerative diseases, neonatal asphyxia, cardiac arrest, cardiogenic shock and acute myocardial infarction, or for use in providing cardioprotection against cardiotoxic drugs, or for use in providing neuroprotection, wherein the components are for administration simultaneously, sequentially or separately.

27. A pharmaceutical product for use according to claim 26 wherein the ischemia and/or reperfusion injury is ischemia and/or reperfusion injury of the brain, heart, lung, kidney, preferably cerebral ischemia, cerebral reperfusion injury or stroke.

28. A pharmaceutical product for use according to any one of claims 26 and 27 wherein the components are for parenteral administration, preferably intravenous administration.

29. A pharmaceutical product for use according to any one of claims 26 to 28 wherein the components are for administration during reperfusion.

30. A pharmaceutical product for use according to any one of claims 26 to 28 wherein the components are for administration before reperfusion.

31. A pharmaceutical product for use according to any one of claims 26 to 28 wherein the components are for administration after reperfusion.

32. A pharmaceutical product for use according to any one of claims 26 to 31 wherein the components are for simultaneous administration.

33. A method of treating and/or preventing one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection, said method comprising simultaneously, sequentially or separately administering to a subject in need thereof:
(a) a sulfonylurea; and
(b) at least one of the following components: (i) an insulin modulator, and (ii) an aldosterone antagonist.

34. A method according to claim 33 which comprises simultaneously, sequentially or separately administering to a subject in need thereof:
(a) glibenclamide, or a structural or functional analogue thereof; and
(b) at least one of the following components: (i) exenatide, or a structural or functional analogue thereof, or a pharmaceutically acceptable salt thereof; and (ii) potassium canrenoate, or a structural or functional analogue thereof.

35. A method according to claim 34 wherein the exenatide structural or functional analogue is a GLP-1 receptor agonist.

36. A method according to any one claims 34 and 35 wherein the exenatide structural or functional analogue is selected from lixisenatide, albiglutide, liraglutide, taspoglutide and dulaglutide (LY2189265).

37. A method according to any one claims 34 to 36 wherein the glibenclamide structural or functional analogue is selected from acylhydrazone, sulfonamide and sulfonylthiourea derivatives of glibenclamide, glimepiride, glipizide and gliclazide, preferably glicazide.

38. A method according to any one claims 33 to 37 wherein the ischemia and/or reperfusion injury is ischemia and/or reperfusion injury of the brain, heart, lung, kidney, preferably cerebral ischemia, cerebral reperfusion injury or stroke.

39. A method according to any one of claims 33 to 38 wherein the components are administered parenterally, preferably intravenously.

40. A method according to any one of claims 34 to 39 wherein the glibenclamide is administered at a dosage of about 0.001 to about 30 μg/kg body weight of the subject.

41. A method according to any one of claims 34 to 39 wherein the exenatide, or pharmaceutically acceptable salt thereof, is administered at a dosage of about 0.001 to about 1.5 μg/kg body weight of the subject.

42. A method according to any one of claims 34 to 39 wherein the potassium canrenoate is administered at a dosage of about 0.03 to about 10 mg/kg body weight of the subject.

43. A method according to any one of claims 33 to 42 which comprises simultaneously administering the components to said subject.

44. Use of:
(a) a sulfonylurea; and
(b) at least one of the following components: (i) an insulin modulator, and (ii) an aldosterone antagonist;
in the manufacture of a medicament for the treatment and/or prevention of one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection.

45. Use of:
(a) glibenclamide, or a structural or functional analogue thereof; and
(b) at least two of the following components: (i) exenatide, or a structural or functional analogue, or a pharmaceutically acceptable salt thereof; and (ii) potassium canrenoate, or a structural or functional analogue thereof;
in the manufacture of a medicament for the treatment and/or prevention of one or more of ischemia and/or reperfusion injury, stroke, neurodegenerative diseases, neonatal asphyxia, cardiac arrest, cardiogenic shock and acute myocardial infarction, or for providing cardioprotection against cardiotoxic drugs, or for providing neuroprotection.

46. A use according to claim 45 wherein the exenatide structural or functional analogue is a GLP-1 receptor agonist.

47. A use according to claim 45 or claim 46 wherein the exenatide structural or functional analogue is selected from lixisenatide, albiglutide, liraglutide, taspoglutide and dulaglutide (LY2189265).

48. A use according to any one claims 45 to 47 wherein the glibenclamide structural or functional analogue is selected from acylhydrazone, sulfonamide and sulfonylthiourea derivatives of glibenclamide, glimepiride, glipizide and gliclazide, preferably glicazide.

49. A use according to any one claims 44 to 48 wherein the ischemia and/or reperfusion injury is ischemia and/or reperfusion injury of the brain, heart, lung, kidney, preferably cerebral ischemia, cerebral reperfusion injury or stroke.

50. A use according to any one of claims 44 to 49 wherein the components are administered parenterally, preferably intravenously.

51. A use according to any one of claims 44 to 50 wherein the components are administered during reperfusion.

52. A use according to any one of claims 44 to 50 wherein the components are administered before reperfusion.

53. A use according to any one of claims 44 to 50 wherein the components are administered after reperfusion.

54. A use according to any one of claims 44 to 53 which comprises simultaneously administering the components to said subject.

55. Use of a combination comprising:
(a) a sulfonylurea; and
(b) at least one of the following components: (i) an insulin modulator, and (ii) an aldosterone antagonist;
for treating and/or preventing ischemia and/or reperfusion injury in an ex vivo organ prior to or during transplantation.

56. Use of a combination comprising:
(a) glibenclamide, or a structural or functional analogue thereof; and
(b) at least two of the following components: (i) exenatide, or a structural or functional analogue thereof, or a pharmaceutically acceptable salt thereof; and (ii) potassium canrenoate, or a structural or functional analogue thereof;
for treating and/or preventing ischemia and/or reperfusion injury in an ex vivo organ prior to or during transplantation.

57. A use according to claim 56 wherein the exenatide structural or functional analogue is a GLP-1 receptor agonist.

58. A use according to claim 56 or claim 57 wherein the exenatide structural or functional analogue is selected from lixisenatide, albiglutide, liraglutide, taspoglutide and dulaglutide (LY2189265).

59. A use according to any one claims 56 to 58 wherein the glibenclamide structural or functional analogue is selected from acylhydrazone, sulfonamide and sulfonylthiourea derivatives of glibenclamide, glimepiride and gliclazide, preferably glicazide.

60. A use according to any one claims 55 to 59 wherein the ischemia and/or reperfusion injury is cerebral ischemia, cerebral reperfusion injury or stroke.

61. A use according to any one of claims 55 to 60 wherein the components are administered prior to transplantation.

62. A combination according to any one of claims 1 to 12 or a pharmaceutical composition according to any one of claims 13 and 14 for use in the treatment and/or prevention of stroke.

63. A combination according to any one of claims 1 to 12 or a pharmaceutical composition according to any one of claims 13 and 14 for use in the treatment and/or prevention of a neurodegenerative disease, preferably selected from Parkinson's disease, Alzheimer's disease, Huntington's disease, amyotrophic lateral sclerosis and vascular dementia.

64. A combination according to any one of claims 1 to 12 or a pharmaceutical composition according to any one of claims 13 and 14 for use in providing neuroprotection.

65. A method according to any one of claims 33 to 43 wherein the neurodegenerative disease is selected from Parkinson's disease, Alzheimer's disease, Huntington's disease, amyotrophic lateral sclerosis and vascular dementia.
